(19) United States
(12) Patent Application Publication
(10) Pub. No.: US 2025/0041330 A1
(43) Pub. Date: Feb. 6, 2025

(54) METHOD OF TREATING OBESITY BY SELECTIVE TARGETING OF VISCERAL ADIPOSITY USING POLYCATION NANOMEDICINE

(71) Applicant: THE TRUSTEES OF COLUMBIA UNIVERSITY IN THE CITY OF NEW YORK, New York, NY (US)

(72) Inventors: Li QIANG, Closter, NJ (US); Kam W. LEONG, New York, NY (US); Baoding HUANG, New York, NY (US); Qianfen WAN, New York, NY (US)

(21) Appl. No.: 18/801,052

(22) Filed: Aug. 12, 2024

Related U.S. Application Data
(63) Continuation of application No. PCT/US2023/062422, filed on Feb. 10, 2023.
(60) Provisional application No. 63/378,782, filed on Oct. 7, 2022.
(60) Provisional application No. 63/309,325, filed on Feb. 11, 2022.

(51) Int. Cl. A61K 31/785; A61K 45/06; A61K 47/28; A61P 3/04

(52) U.S. Cl. CPC A61K 31/785; A61K 45/06; A61K 47/28; A61P 3/04

(57) ABSTRACT

The method of treating obesity and targeting adipose tissue in a patient may use injection of a polycationic polymer, such as polyamidoamine (PAMAM) generation 3 (P-G3) or a PCL-g-PAMAM Denpol, that when delivered intraperitoneally, selectively targets visceral adiposity due to its high charge density. P-G3 treatment of obese mice inhibits visceral adiposity, increases energy expenditure, prevents obesity, and alleviates associated metabolic dysfunctions. The extracellular matrix of adipose tissue is enriched with glycosaminoglycans, the known biomacromolecules with the strongest negative charge. P-G3 uncouples adipocyte lipid synthesis and storage from adipocyte development, creating adipocytes with normal functions but that are deficient in hypertrophic growth. The visceral fat distribution of P-G3 is further enhanced by modifying P-G3 with cholesterol to form lipophilic nanoparticles, effective in treating obesity. This strategy provides a method to target visceral adiposity, and cationic nanomaterials useful for treating metabolic diseases or for delivery of additional treating agents.

### CROSS-REFERENCE TO RELATED APPLICATIONS

This application is a continuation of International Application No. PCT/US2023/062422, filed on Feb. 10, 2023, which claims benefit of U.S. Provisional Application No. 63/309,325 filed Feb. 11, 2022, and U.S. Provisional Application No. 63/378,782 filed Oct. 7, 2022, the contents of which are hereby incorporated by reference.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT LICENSE RIGHTS

This invention was made with government support under grant no. AR073935, awarded by the National Institutes of Health (NIH). The government has certain rights in the invention.

### BACKGROUND

### 1. Field

The disclosure of the present patent application relates to the treatment of obesity and other conditions by the targeting of adipose tissue, such as visceral adiposity, and particularly to the intraperitoneal administration of polycationic polymers, such as polyamidoamine (PAMAM) generation 3 (P-G3), or of cationic dendronized polymers (cDenpols) including polycaprolactone (PCL) as the backbone, such as PCL-g-PAMAM Denpols.

### 2. Description of the Related Art

Obesity and overweight are surging global health challenges, inflicting severe comorbidities including diabetes and cardiovascular diseases to account for the second most preventable death 1. Obesity is directly caused by the expansion of white adipose tissue (WAT), owing to the formation and growth of adipocytes. Adipocytes function by storing lipids in the form of triglycerides (TG). The size of an adipocyte can grow up to 20-fold in diameter, theoretically holding ˜8,000-fold more lipids². Of note, the metabolic risks of obesity depend largely on body fat distribution rather than excess weight per se. WAT can be mainly classified as subcutaneous or visceral fat according to the anatomical location 3. The former is underneath the skin, whereas the latter localizes inside the peritoneal cavity and is more robustly associated with obesity comorbidities 4. Nevertheless, obesity treatment remains a significant challenge, particularly for visceral adiposity.

Cationic nanomaterials, represented by PAMAM dendrimers, have shown promising potential in treating various inflammatory diseases and cancers through neutralizing negatively charged pathogens⁵⁻⁸ but have never been applied to obesity.

During the progression of obesity, the expansion of WAT is accompanied by increased production of extracellular matrix (ECM)⁹, which contains glycosaminoglycans (GAGs), the known biomacromolecules with the strongest negative charge¹⁰. The anionic nature of ECM in adipose tissue suggests that cationic nanomaterial would be enriched in this tissue. P-G3 is a third-generation PAMAM dendrimer with 32 surface amine groups (MW 6,909, size 36 Å)¹¹.

Here, we report that P-G3 and its lipophilic derivative are preferentially distributed to visceral fat and inhibit diet-induced obesity (DIO) in a murine model, enlightening a polycationic strategy to tackle visceral adiposity.

During the progression of obesity, the expansion of adipose tissue is accompanied by increased production of extracellular matrix (ECM), which contains glycosaminoglycans (GAGs), the known biomacromolecules with the strongest negative charge. Considering the anionic nature of ECM in adipose tissue, cationic macromolecules could be selectively enriched in this tissue. It would be desirable to treat obesity or other conditions through targeting adipose tissue, through administration of polycationic polymers such as P-G3, preferentially distributing to visceral fat, for example to prevent diet-induced obesity (DIO).

Obesity is placing a tremendous challenge to health care system worldwide. According to the World Health Organization's (WHO's) data, the worldwide prevalence of obesity nearly tripled from 1975 to 2016. Obesity is associated with the development of metabolic syndrome (MetS), which is defined as 3 of 5 risk factors (excess abdominal fat, hypertriglyceridemia, low HDL-C, hypertension, and hyperglycemia) by the Harmonized Definition. The overall prevalence of MetS in adults older than 20 years in the United States surged to 35% from 2011 to 2016. The high prevalence is particularly alarming given that obesity and MetS also predispose to a number of serious conditions such as T2DM, CVD, osteoarthritis, various types of cancer, among others. All these spectra of diseases contribute to huge increase of morbidity and mortality in modern society.

It is also well recognized that obesity is associated with chronic low-grade inflammation condition, which is defined as metabolic inflammation or “meta-inflammation” involving a variety of tissues like AT (adipose tissue, also known as fat tissue or fatty tissue), skeletal muscle, liver, as well as the vascular and immune systems. The signals that initiate and maintain these inflammatory changes are not well known and may include multiple factors like dietary fatty acid, hypoxia, and endotoxin. It plays a causal role in the development of insulin resistance and T2DM.

It is evident that sustained weight loss is a straightforward treatment option for associated metabolic disorders. Unfortunately, it is far from easy to maintain weight loss with current approaches. Because of the role connecting obesity and the related comorbidities, targeting inflammation represents a new therapy for obesity-linked diseases. Although achieving benefit in animals, the results of clinical trials with anti-inflammatory drugs have so far been frustrating. It is therefore essential to develop new targets or strategies to treat obesity-associated metabolic diseases, and otherwise to target adipose tissue, such as for drug delivery.

Polyamidoamine (PAMAM) is the most well characterized class of nanoscale dendrimer, possessing a symmetric and highly branching molecular structure. In recent years, it has gained attention as a promising candidate in nanomedicine. Other than its conventional use as carrier for drug or gene delivery, cationic PAMAM additionally functions by neutralizing the negatively charged pathogens, such as cell-free nucleic acids (cfNAs) released by dead and dying cells, sometimes contributed by gut microbiome, virus, or even food. It has been shown that cationic polymer or nanoparticles as scavenger can produce an improvement in animal disease models involved in acute inflammation, including acute liver failure, trauma, lupus, rheumatoid arthritis, and bacterial sepsis.

PCL-g-PAMAM Denpols have been demonstrated as molecular scavengers. Peng, B., et al, Tuned Cationic Dendronized Polymer: Molecular Scavenger for Rheumatoid Arthritis Treatment, Angew. Chem. Int. Ed., vol. 58, pages 4254-58 (2019).

There has been no report that these polycationic materials would be able to mitigate metabolic disorders with the feature of meta-inflammation, which lack for effective treatment to date. Thus, a method of treating obesity solving the aforementioned problems, or otherwise targeting adipose tissue, including for delivery of treating agents, is desired.

### SUMMARY

The present method of treating obesity targets visceral adiposity in a patient by intraperitoneally injecting polycationic polymer polyamidoamine (PAMAM) generation 3 (P-G3) into the patient. The extracellular matrix of adipose tissue is enriched with glycosaminoglycans, the known biomacromolecules with the strongest negative charge. P-G3 selectively accumulates in the visceral fat through electric charge affinity when delivered intraperitoneally. P-G3 improves adipose remodeling, inhibits visceral adiposity, and prevents obesity in a murine model. In vitro adipocyte models and single-cell RNA sequencing (scRNA-seq) revealed that P-G3 uncouples the defining function of adipocyte-lipid synthesis and storage from the adipocyte development stage to create unique “dwarf” adipocytes possessing normal adipocyte functions but specifically deficient in hypertrophic growth. The visceral fat distribution of P-G3 is further enhanced by engineering with cholesterol to form lipophilic nanoparticles, which showed therapeutic potential in treating obesity.

As a prototypical scavenger, P-G3 is shown to have an unexpectedly extraordinary weight loss effect in DIO mice, accompanied with improved metabolic status. Meanwhile, the elevated cfRNA in an obese sample group was ameliorated in line with the ease of TLR3. After PG3 treatment, cfRNA is decreased in the circulation, while the decreased activation of TLR3 is also observed.

In order to treat, prevent, or help reduce obesity or its onset, a pharmaceutically acceptable amount of polycationic polymer polyamidoamine (PAMAM) generation 3 (P-G3) may be administered to a patient in need thereof. As a non-limiting example, the administration of the P-G3 may be performed by intraperitoneal (IP) injection. Further, the cationic nanomaterial may be administered in the form of lipophilic P-G3 nanoparticles when cholesterol moieties are conjugated to P-G3. Moreover, the P-G3-cholesterol nanoparticles can encapsulate anti-obesity drugs to further potentiate the treatment efficacy, or otherwise act as drug carriers for targeted delivery of a treating agent to adipose tissue. This method may be used to treat, for example, obesity, diabetes, liver metabolic diseases, and aging. Liver metabolic diseases include, without limitation, Nonalcoholic fatty liver disease (NAFLD), nonalcoholic steatohepatitis (NASH), hereditary hemochromatosis, Alpha-I Antitrypsin Deficiency (AATD), and Wilson Disease.

Similarly, PCL-g-PAMAM Denpols may be used for these purposes.

These and other features of the present subject matter will become readily apparent upon further review of the following specification.

### BRIEF DESCRIPTION OF THE DRAWINGS

FIG. 1A-FIG. 1I: P-G3 is selectively distributed to visceral fat.

FIG. 1A, Schematic of the P-G3 structure and adipose tissue with ECM and ex vivo imaging of Cy5 fluorescent signal of tissues after incubating with Cy5-labelled P-G3 for 45 min. All the tissues were imaged at the same exposure.

FIG. 1B, Schematic of P-G3 administration and tissue distribution. The outer ring represents distribution preference (created with BioRender.com).

FIG. 1C, In vivo imaging of P-G3 signal at 8, 24 and 48 h post i.p. injection. PBS-treated (vehicle) mice were used as the basal reference.

FIG. 1D. Imaging of signal intensity in individual tissues from mice sacrificed at 80 h post-injection.

FIG. 1E, Confocal microscopy analysis of P-G3 distribution in eWAT, iWAT and liver from mice at 80 h after Cy5-labelled P-G3 injection,

FIG. 1F, Colocalization of Cy5-labelled P-G3 with DAPI (staining for nuclei) and caveolin-1 (staining for adipocyte cell membrane) in eWAT, Representative data in e and f were independently repeated twice with similar results.

FIG. 1G, Structural illustrations of P-G3, branched polyethylenimine (B-PEI), linear polyethylenimine (L-PEI) and polyanionic P-G2.5.

FIG. 1H, FIG. 1I, Tissue distribution of Cy5-labelled P-G3, B-PEI and L-PEI (H) and P-G2.5 (I) at 72 h post-injection. The same tissues were used for the PBS and P-G3 groups. The unit of the fluorescent scale bar is photons s⁻¹ cm⁻² sr⁻¹.

FIG. 2A-FIG. 2O: P-G3 prevents DIG and improves metabolic health.

FIG. 2A, Schematic of the experimental design (created with BioRender.com).

FIG. 2B, Representative mouse pictures at sacrifice after eight-week treatment,

FIG. 2C, Body weight curve before disturbance by metabolic measurements.

FIG. 2D, Average food intake of five continuous days on HFD feeding.

FIG. 2E, Faecal free fatty acid content (separate cohort of mice, n=7 versus 7).

FIG. 2F, Body composition determined by EchoMRL.

FIG. 2G, Tissue weights at sacrifice,

FIG. 2H-FIG. 2K, Histological analysis (H&E staining) and adipocyte size distribution and gene expression of eWAT (H and J) and iWAT (I and K).

FIG. 2L, FIG. 2M, Male mice received three dosages of P-G3 (10 mg per kg of body weight) or vehicle (i.p.) twice weekly since the beginning of HFD feeding and then housed singly in metabolic cages for calorimetric analysis (n=7 versus 7). Energy expenditure indicated by correlating heat production to lean body mass (L). O₂ consumption (M).

FIG. 2N, FIG. 2O, GTT (N) and ITT (O) in mice at 6.5 and 6.0 weeks of P-G3 treatment (same cohort as in a). For FIGS. 2C, D, F-I, N and O, data are presented as mean±s.e.m. (n=8 for vehicle group; n=5 for P-G3 group). For FIGS. 2J and K, data are presented as mean±s.e.m. (n 6 for vehicle group; n 5 for P-G3 group). For FIGS. 2E, L and M, data are presented as mean±s.e.m. n=7 for vehicle group; n=7 for P-G3 group). Statistical significance is calculated via a two-tailed Student's t-test.

FIG. 3A-FIG. 3H: P-G3 uncouples lipid synthesis from adipocyte formation.

FIG. 3A-FIG. 3D, 3T3-L1 or C3H10T1/2 preadipocytes were differentiated in the presence of 10 μg ml⁻¹ P-G3, Oil Red O staining of lipid droplets in 3T3-L1 cells on day 6 of differentiation (A), BODIVY staining of lipid droplets and the quantification of lipid droplet size in C3H10T1/2 cells on differentiation day 12 (B). The representative data in a and b were independently repeated three tines with similar results. Figs. C, D, qPCR analysis of the gene expression of adipogenic markers (C) and lipogenic genes (D) during the time course of 3T3-L1 differentiation with or without P-G3 treatment. The data are represented as mean±s.e.m. (n=4, 4). Statistical significance is calculated via a two-tailed Student's t-test.

FIG. 3E, Schematic of the P-G3's bifurcate regulation of adipocyte development (created with BioRender.com).

FIG. 3F, qPCR analysis of gene expression in mature 3T3-L1 adipocytes after treatment with 10 μg ml⁻¹ P-G3 from day 9 to day 14, The data are represented as mean±s.e.m. (n=4, 4). Statistical significance is calculated via a two-tailed Student's t-test.

FIG. 3G, qPCR analysis of expression of the genes involved in lipid metabolism in eWAT from HFD mice after eight-week P-G3 treatment. The data are represented as mean±s.e.m. (n=6, 5), Statistical significance is calculated via a two-tailed Student's t-test.

FIG. 3H, qPCR analysis of expression in ex vivo human omental adipose tissue after treatment with 10 μg ml⁻¹ P-G3 for eight days. The data are represented as mean±s.e.m. (n=4, 3). Statistical significance is calculated via a two-tailed Student's t-test.

FIG. 4A-FIG. 4I: scRNA-seq analyses reveal the bifurcate regulation of adipocyte development by P-G3.

FIG. 4A, RNA velocity revealed the cell dynamics during 3T3-L1 differentiation.

FIG. 4B, Schematic of the heterogeneity of adipogenesis, including preadipocyte, immature adipocyte, adipocyte and lipogenic adipocyte.

FIG. 4C, Cell-type composition of the cells on day 3 and day 6 of differentiation.

FIG. 4D, Clustering of adipocytes shown in the uniform manifold approximation and projection space colored at different time points during adipogenesis with or without P-G3 treatment.

FIG. 4E, Expression of spliced mature mRNA of Pparg gene.

FIG. 4F, RNA velocity of Pparg gene based on unspliced/spliced mRNA ratio,

FIG. 4G, Expression of spliced mature mRNA of Fasn gene showed the cell population as lipogenic adipocytes,

FIG. 4H, RNA velocity of Fasn gene based on unspliced/spliced mRNA ratio.

FIG. 4I, Dot plot showing the key altered pathways on day 6 by QIAGEN IPA two-sided analysis. The dot sizes and color reflect the p values (shown in −log 10 (p value)) in each pathway.

FIG. 5A-FIG. 5G: P-G3 represses mTOR signaling pathway and decreases NAD⁺ levels in adipocyte development.

FIG. 5A, Colocalization of Cy5-labelled P-G3 with DAPI and LysoTracker in 3T3-L1 matured adipocytes after 24 h of P-G3 treatment. The representative data are independently repeated twice with similar results.

FIG. 5B, Flow cytometry determination of lysosomal intracellular activity of C3H10T1/2 cells treated with P-G3 or bafilomycin A1. Cells without self-quenched substrate incubation (grey curve) were used as the baseline reference.

FIG. 5C, Western blot analysis of the mTOR pathway during 3T3-L1 differentiation in the presence or absence of P-G3. The representative data are independently repeated three tines with similar results.

FIG. 5D, Western blot analysis of the mTOR pathway in the eWAT of P-G3-treated mice and quantification. The data are represented as mean±s.e.m. (n=4, 5). Statistical significance is calculated via a two-tailed Student's t-test.

FIG. 5E, qPCR analysis of lipogenic genes in 3T3-L1 adipocytes after rapamycin or P-G3 treatment from day 4 to day 6. The data are represented as mean±s.e.m. (n=4, 4). Statistical significance is calculated via a two-tailed Student's t-test (treatment group versus vehicle group).

FIG. 5F, Decrease in cellular NAD levels in 3T3-L1 preadipocytes after 10 μg ml⁻¹ P-G3 treatment for 14 h. The data are represented as mean±s.e.m. (n=3, 3), Statistical significance is calculated via a two-tailed Student's t-test.

FIG. 5G. Gene expression of 3T3-L1 cells treated with 10 μg ml⁻¹ P-G3, or P-G3 (10 μg ml⁻¹) and NMN (20 mM) at day 0-3 during differentiation. The data are represented as mean±s.e.m. (n=4, 4). Statistical significance is calculated via a two-tailed Student's t-test (treatment group versus vehicle group).

FIG. 6A-FIG. 6L: Engineering P-G3 to improve visceral fat distribution and treat obesity.

FIG. 6A, Structure of adding a 5× cholesterol tail to P-G3 to generate P-G3-Chol(5), which further self-assembles in water and forms spherical NPs.

FIG. 6B, Electronic microscopy of P-G3-Chol(5) NPs and characterizations. The representative data are independently repeated twice with similar results.

FIG. 6C, 200 μg Cy5-labelled NPs or Cy5-labelled P-G3 were i.p. injected into mice and IVIS determination of tissue distribution at 72 h post-injection.

FIG. 6D, Schematic of the experimental design for obesity treatment.

FIG. 6E, Representative mouse pictures after six weeks of NPs treatment.

FIG. 6F, Body weight curve before disturbance by metabolic measurements.

FIG. 6G, Body composition changes during the treatment.

FIG. 6H, Tissue weights at sacrifice.

FIG. 6I, Histological analysis (H&E staining) and adipocyte size distribution of eWAT.

FIG. 6J, FIG. 6K, qPCR analysis of gene expression of adipogenic (J) and lipogenic (K) markers in eWAT after six-week treatment. The data are represented as mean±s.e.m. (n=8, 8). Statistical significance is calculated via a two-tailed Student's t-test.

FIG. 6L, GTT of mice at 5 weeks post-injection. For Figs. D-I and L, n=10, 10, except for h eWAT (n=9, 10). The data are represented as mean±s.e.m. The unit of the fluorescent scale bar is photons s⁻¹ cm⁻² sr⁻¹.

Similar reference characters denote corresponding features consistently throughout the attached drawings.

### DESCRIPTION OF THE EMBODIMENTS

In a method for treating or preventing obesity or its onset, or otherwise targeting adipose tissue, a pharmaceutically acceptable amount of polycationic polymer, such as polyamidoamine (PAMAM) generation 3 (P-G3), or a PCL-g-PAMAM Denpol, may be administered to a patient. As a non-limiting example, the administration of the P-G3 may be performed by intraperitoneal (IP) injection. Further, the P-G3 may be administered in the form of lipophilic P-G3 nanoparticles. The polycationic polymer may be used as a drug carrier, targeting delivery of a treating agent to adipose tissue.

### P-G3 is Selectively Distributed to Visceral Fat

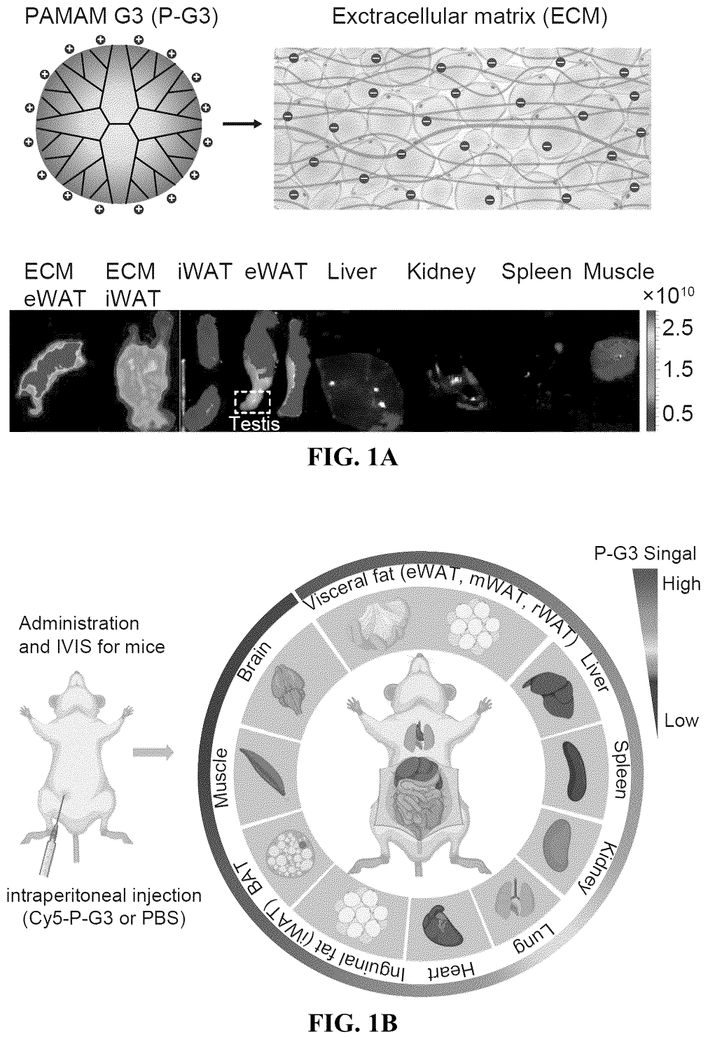

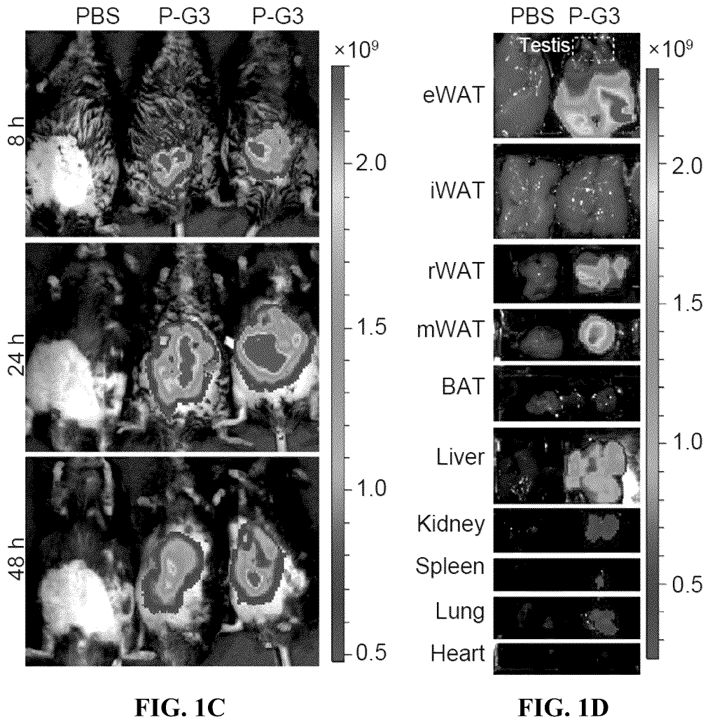

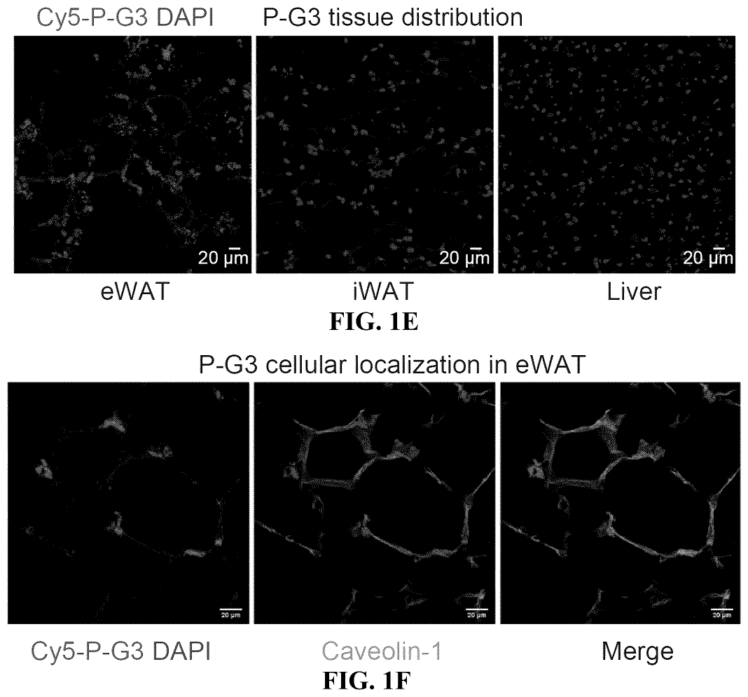

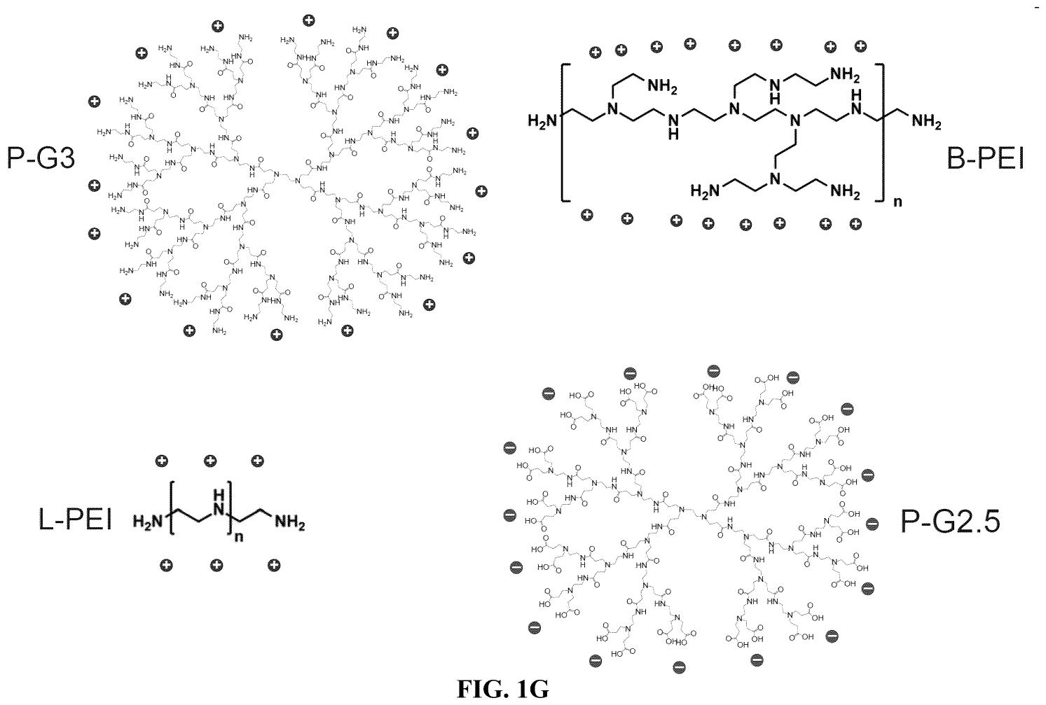

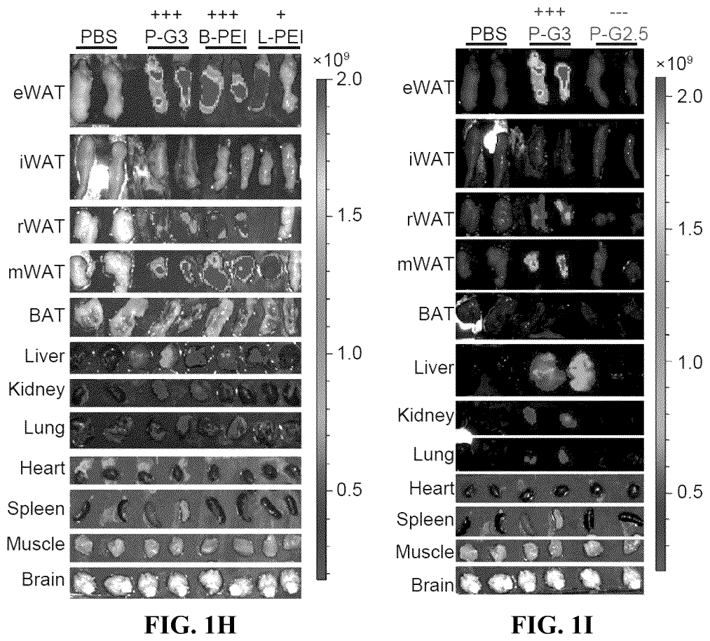

Leveraging the highly anionic ECM in adipose tissue, we tested whether P-G3 can bind to adipose tissue (FIG. 1A and Extended Data FIG. 1a¹). Isolated ECM from visceral epididymal (eWAT) and subcutaneous inguinal (iWAT) adipose tissues showed strong absorption of Cy5 fluorescent-labeled P-G3. Further, ex vivo incubation of P-G3 with intact organs showed a much stronger fluorescent signal in iWAT and eWAT than in non-adipose tissues, without detection in the attached testis (FIG. 1A). These data imply a preferential biodistribution of P-G3 into WAT. ¹ References to Extended Data Figures and Supplementary Tables refer to the “extended data figures” and “supplementary tables” associated with and found in the publication, Wan, Q., et al., “Selective targeting of visceral adiposity by polycation nanomedicine,” Nature Nanotechnology, Vol. 17, pp. 1311-1321 (Dec. 1, 2022),⁴² the contents of which are hereby incorporated by reference in their entirety.

In DIO mice, Cy5-P-G3 signals were enriched in the peritoneal region and peaked at 24 hr post-injection via the regular intraperitoneal (i.p.) delivery route (FIGS. 1B, 1C). At the organ level, the strongest fluorescent signals were detected in all visceral fat depots, including eWAT, mesenteric (mWAT), and retroperitoneal (rWAT), in striking contrast to the minimal signals in iWAT and interscapular BAT (FIG. 1D and Extended Data FIG. 1b). Low levels of Cy5 signal were detected in the liver, lung, spleen, and kidney, indicating that P-G3 entered circulation and was absorbed by other tissues (FIG. 1D and Extended Data FIG. 1b). Histological analyses of tissue sections confirmed the more robust signal in eWAT but not in iWAT and liver (FIG. 1E). Importantly, the P-G3 Cy5 signal evenly penetrated into eWAT rather than attached to the tissue surface. Moreover, colocalization of the P-G3 Cy5 signal and adipocyte marker Caveolin-1 was detected (FIG. 1F), confirming entering into adipocytes. This tissue distribution pattern was recapitulated in chow-fed lean mice (Extended Data FIG. 1c-e). No signal was detected in any adipose tissue by intravenous injection, but subcutaneous fat injection retained the signal locally without distributing into visceral fat despite marginal deposition into other organs (Extended Data FIG. 1f), denoting a diet-independent and delivery route-dependent visceral fat distribution of P-G3.

To understand this visceral fat distribution of P-G3, we manipulated cationic polymers regarding charge and structure by employing branched polyethylenimine (B-PEI) (MW: 25,000) and linear polyethylenimine (L-PEI) (MW: 2,500, weaker cationic charges) (FIG. 1G). Compared with P-G3, B-PEI showed similar preferential distribution to visceral fat depots, whereas L-PEI still displayed overall fainter but still visceral fat preference (FIG. 1H and Extended Data FIG. 1b). Furthermore, polyanionic PAMAM G2.5 (P-G2.5), which has the same structure as P-G3 but is sodium carboxylate terminated (FIG. 1G), showed no signal in any of the tissues (FIG. 1I). These data together indicate that a cationic character is essential for selective visceral fat distribution, and the selectivity can be further tuned by charge density and molecular structure.

### P-G3 Inhibits Diet-Induced Obesity

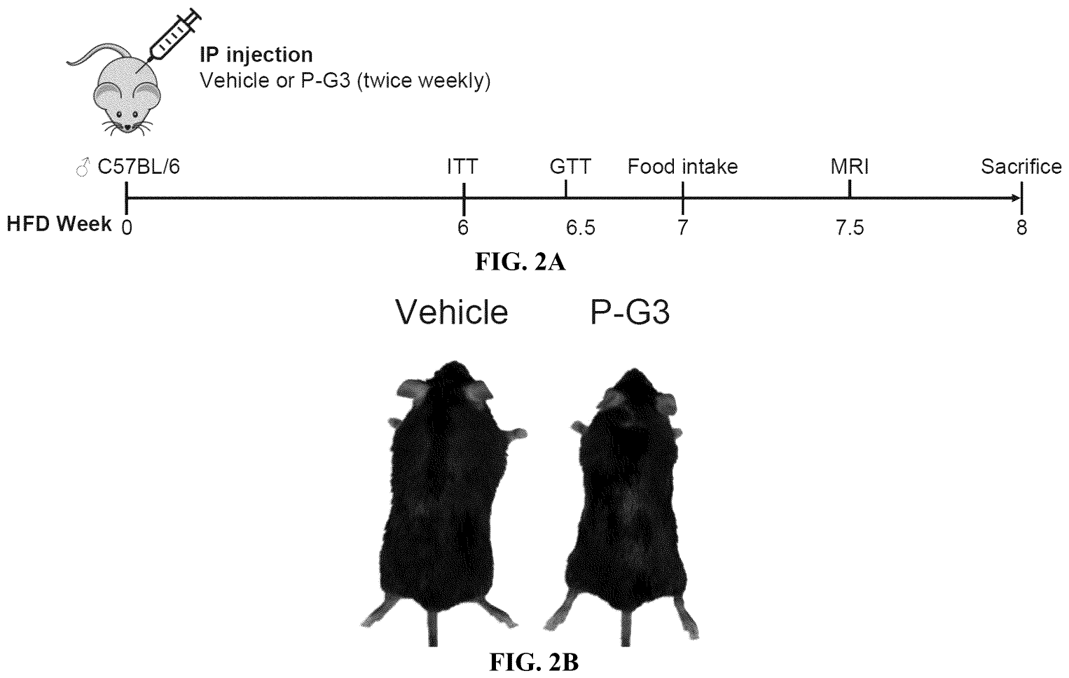

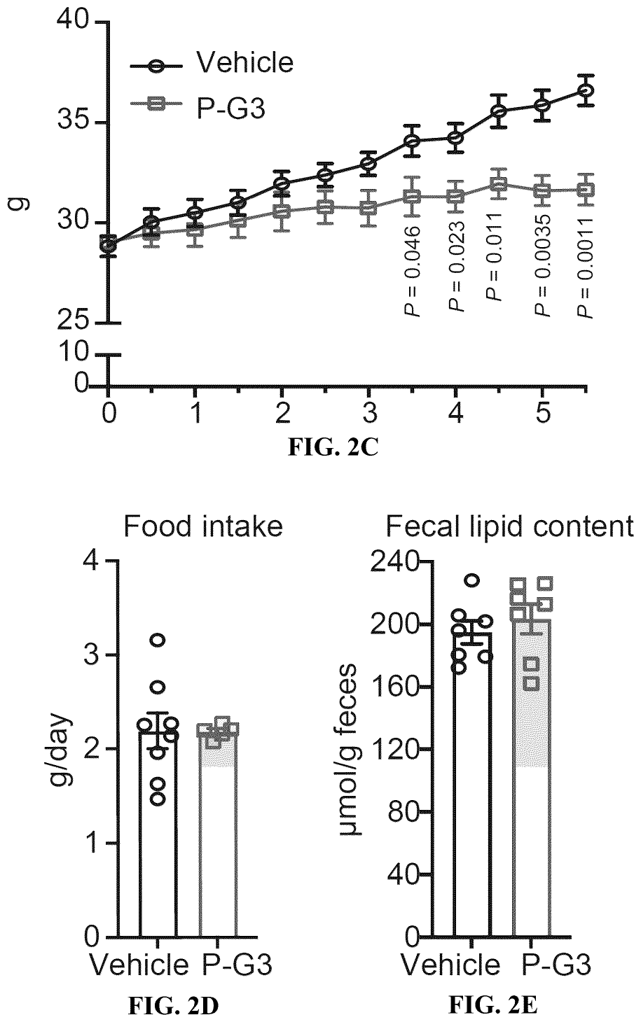

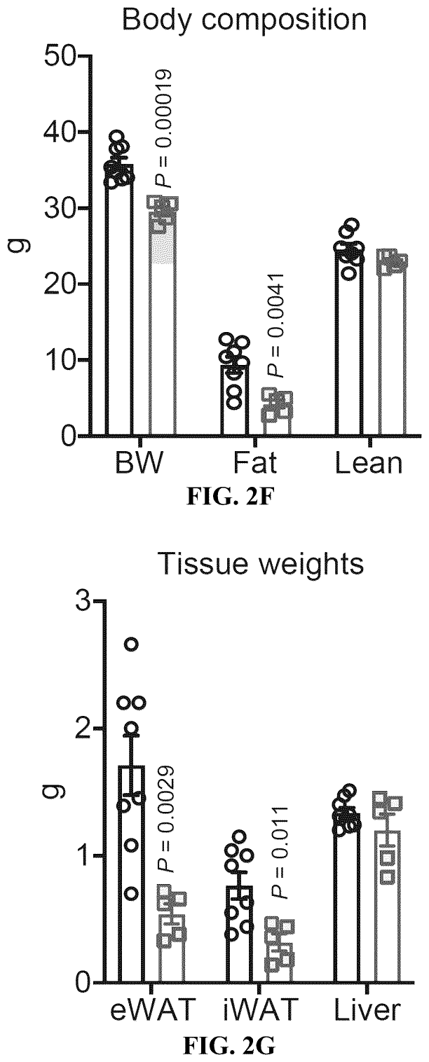

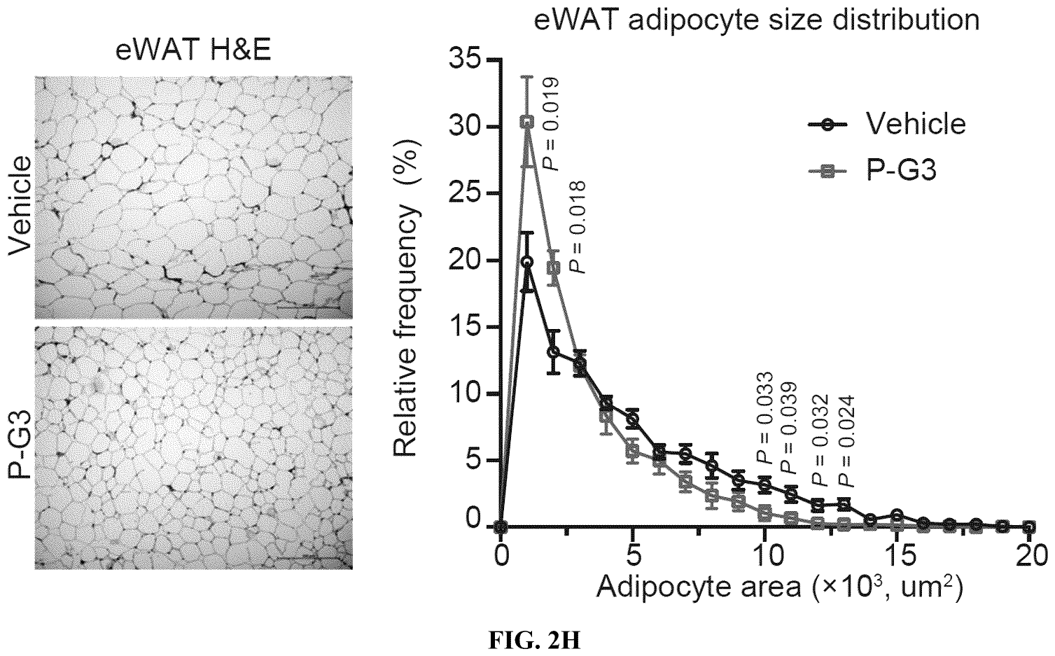

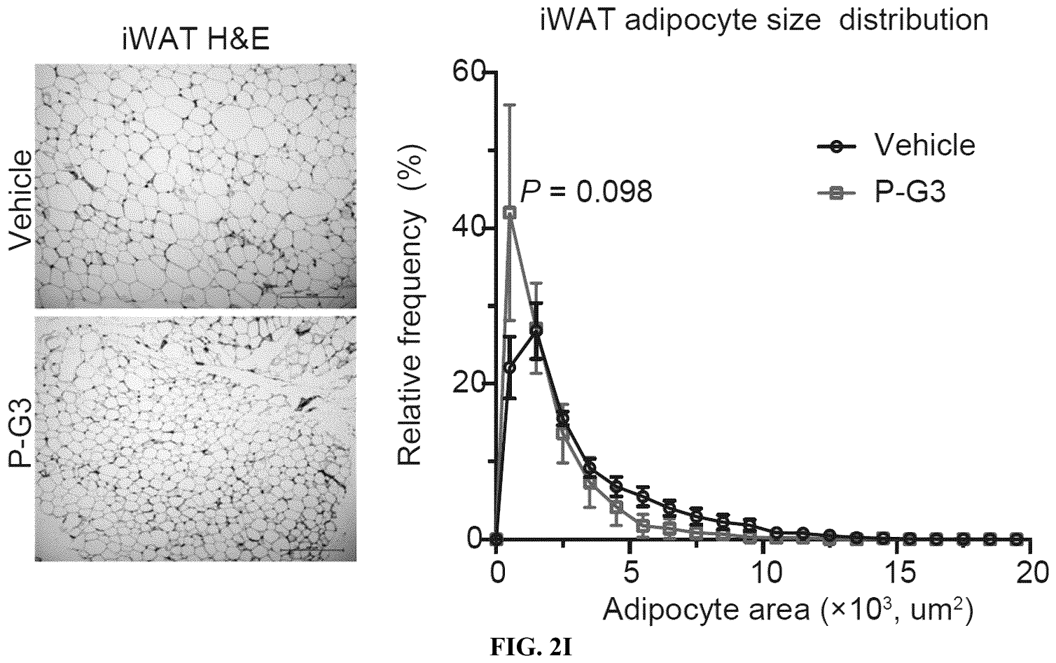

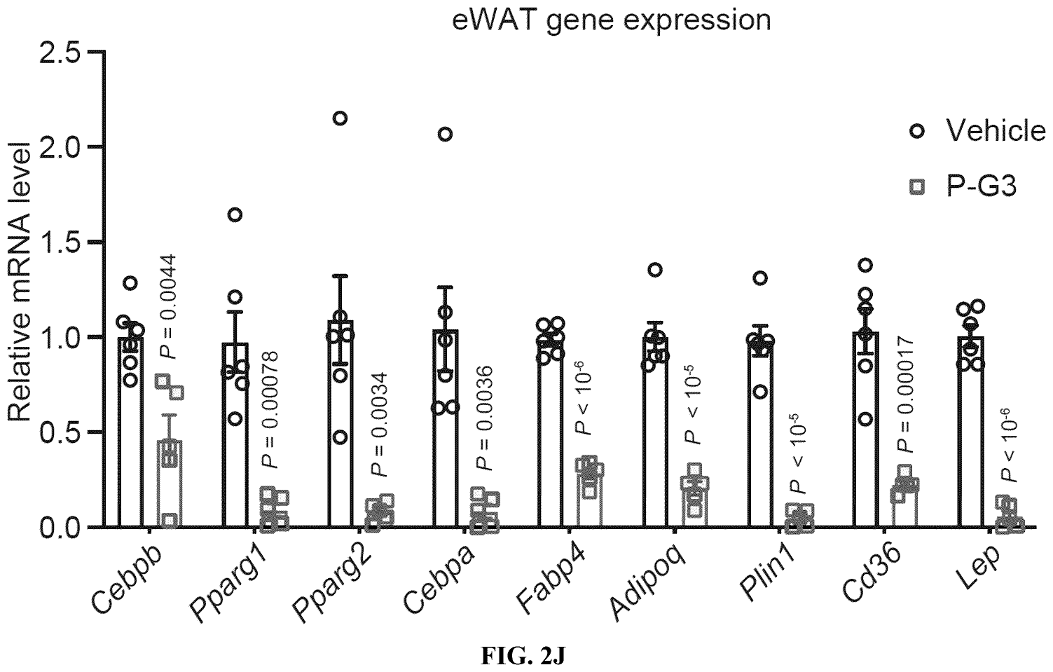

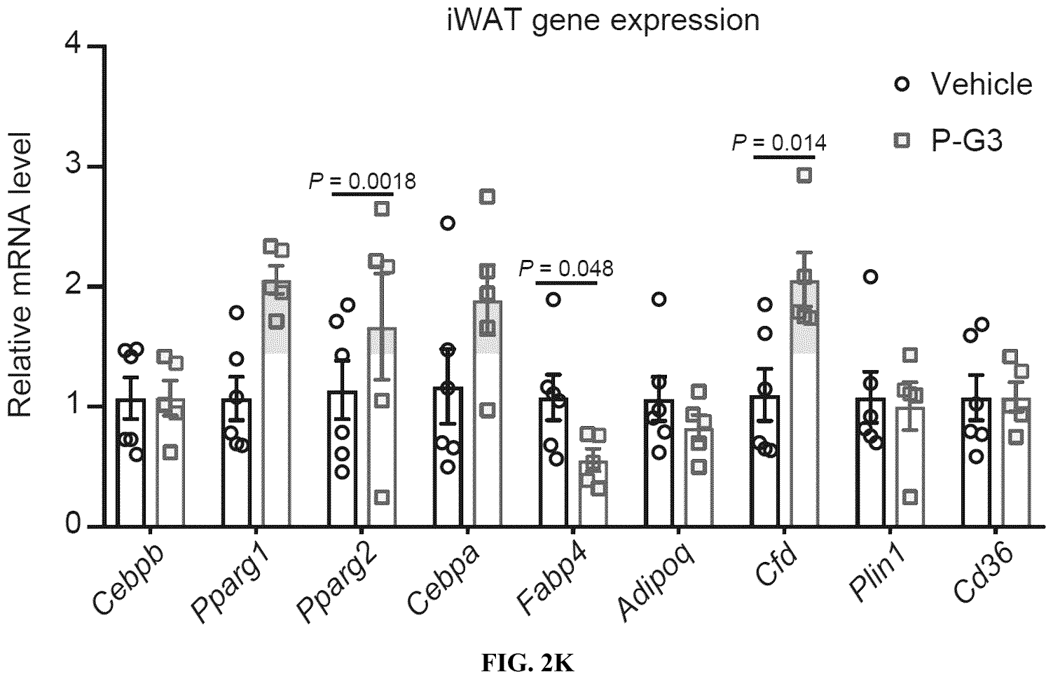

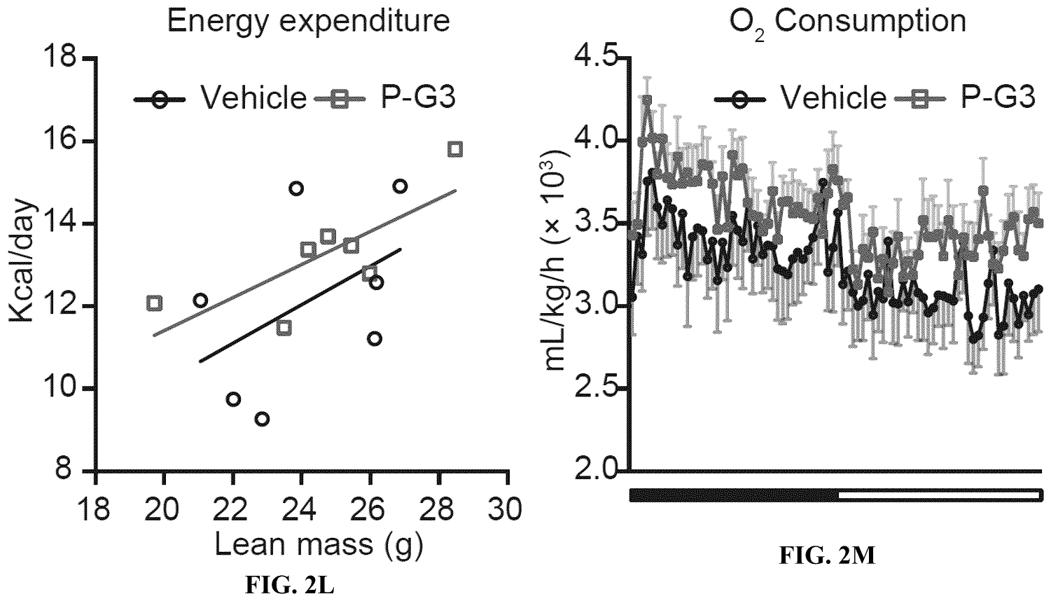

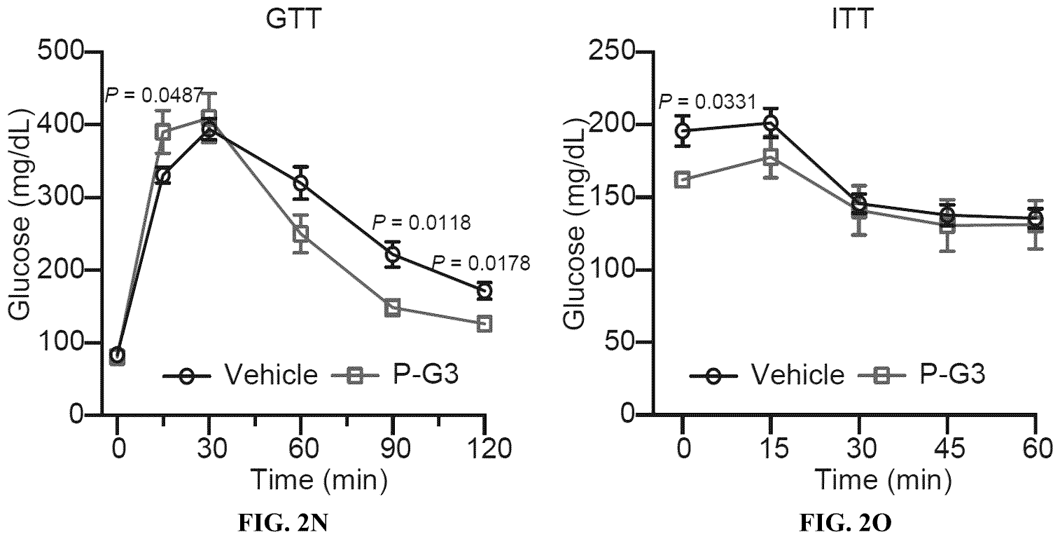

Administering P-G3 nearly completely flattened the steep weight gain on high-fat diet (HFD) feeding, resulting in 20% less body weight after 6-week treatment (FIG. 2A-C). This anti-obesity effect was not caused by reducing food intake (FIG. 2d), intestinal absorption (FIG. 2E), or lean mass (FIG. 2F). Instead, it was exclusively accounted for by inhibiting fat mass (FIG. 2F). This inhibition was more pronounced on the eWAT than on the iWAT, reduced by 70% and 50%, respectively (FIG. 2G). The inhibition of weight gain was recapitulated in female mice (Extended Data FIG. 2a-d) but blunted in chow-fed lean mice (Extended Data FIG. 2e-g), reinforcing the anti-obesity effect of P-G3.

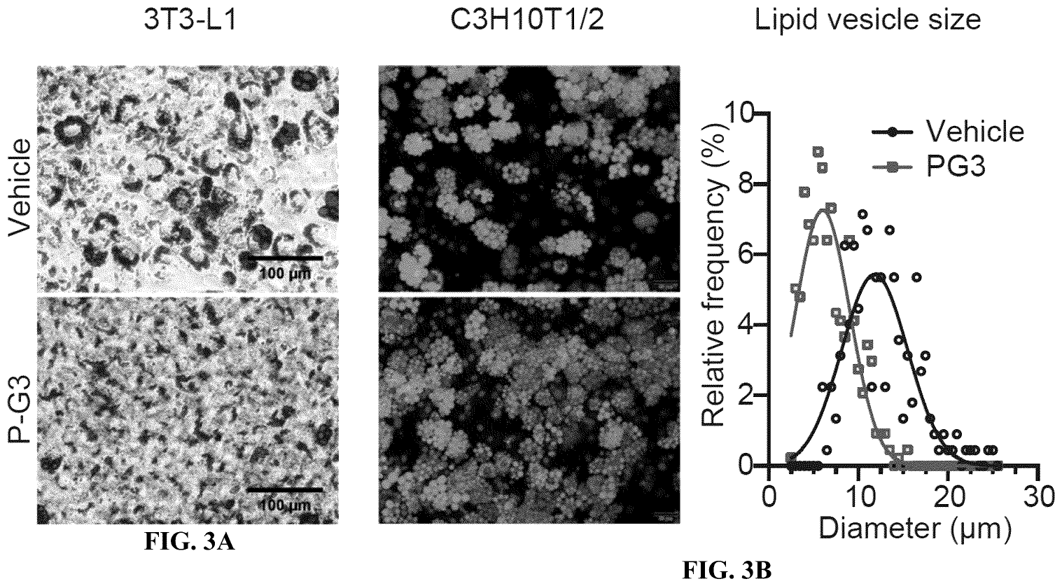

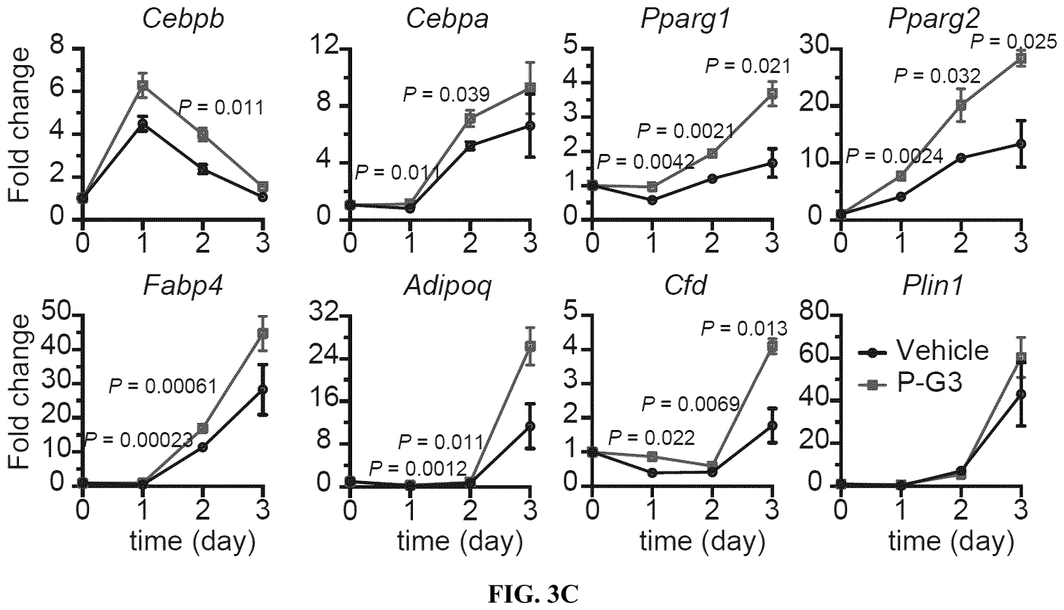

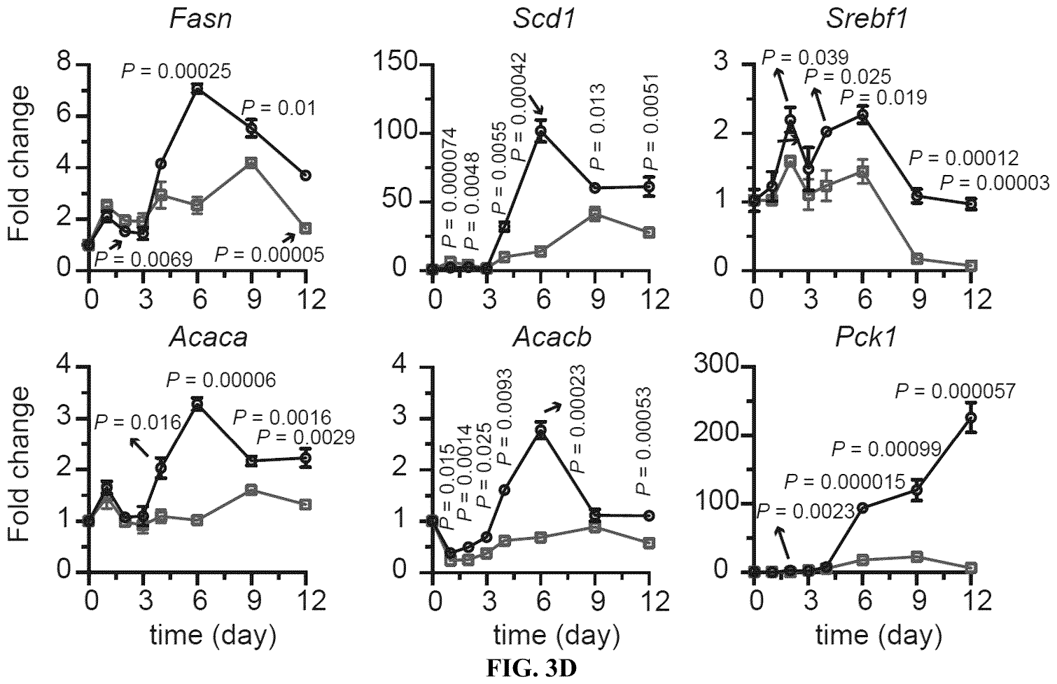

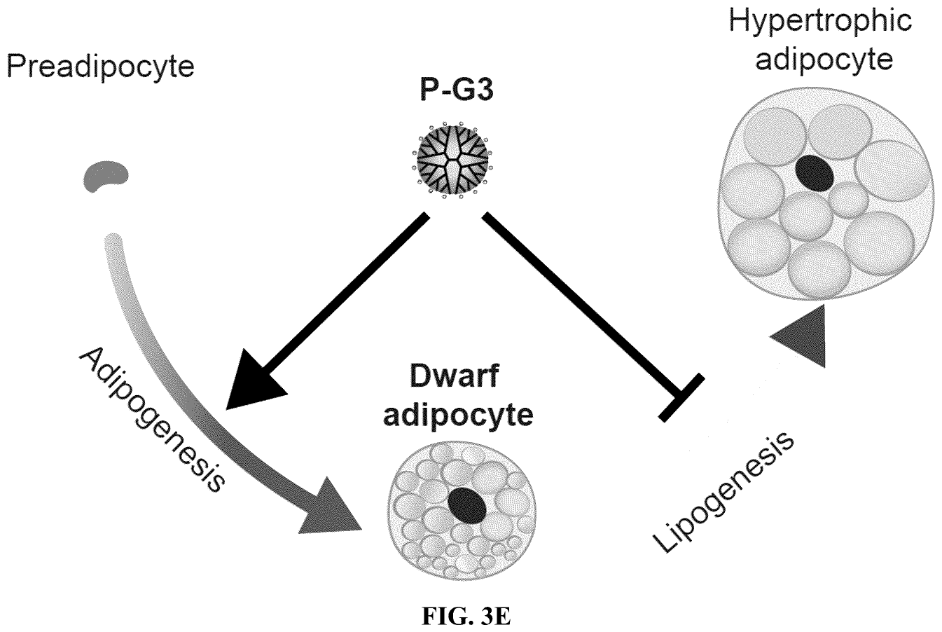

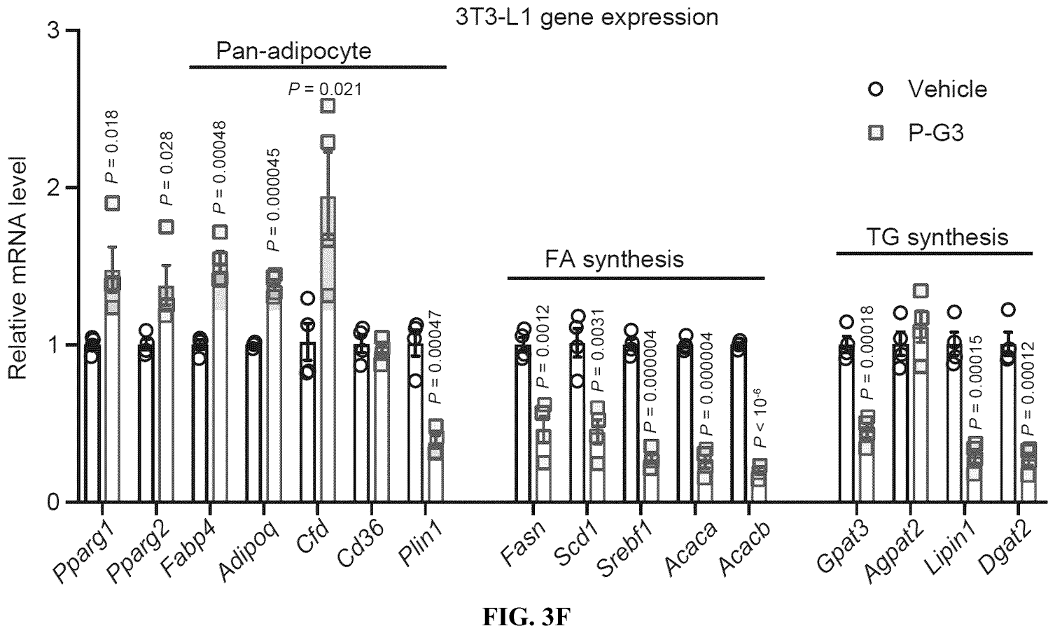

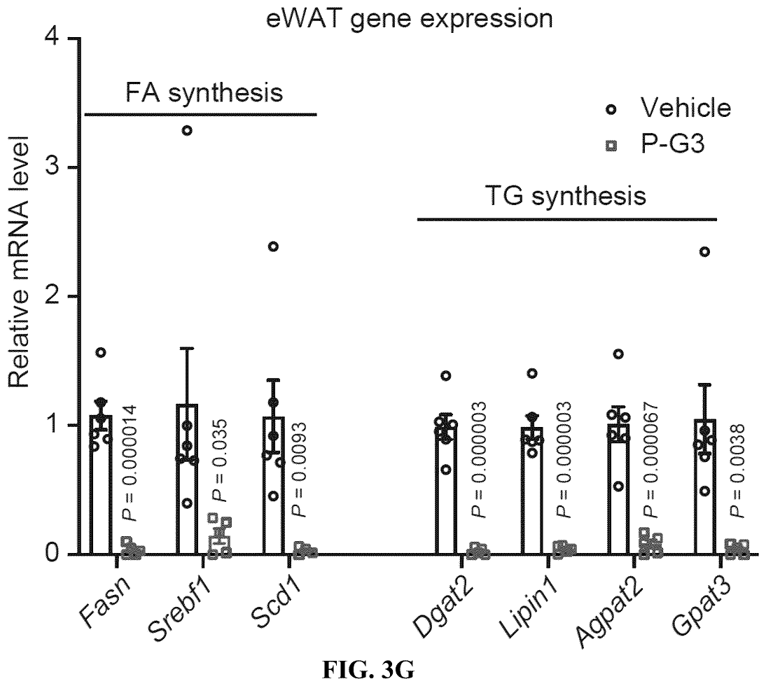

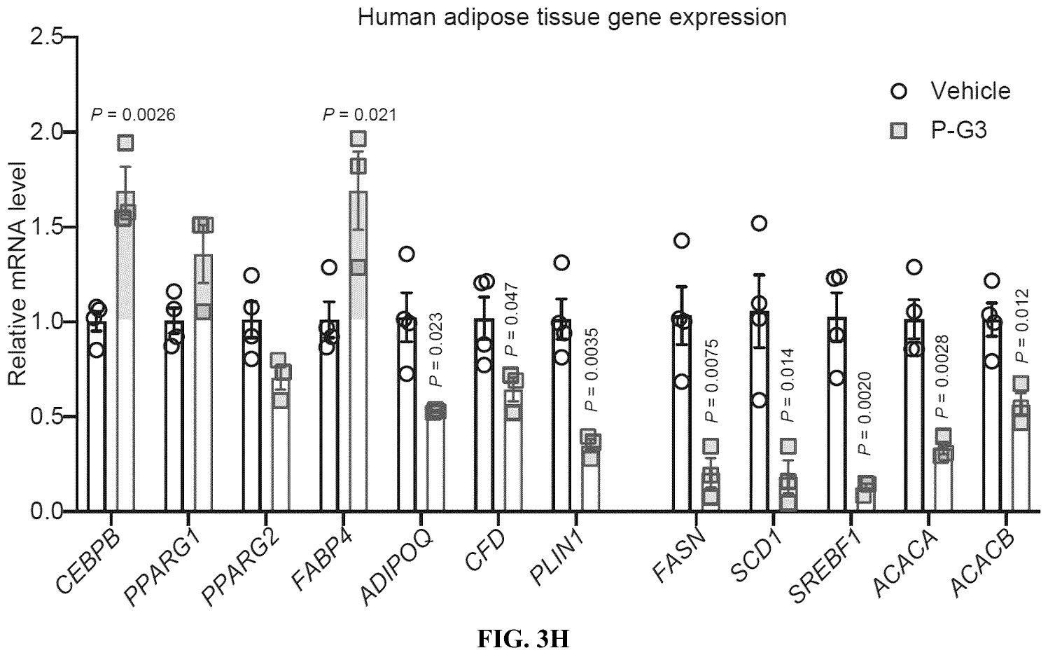

Adipocyte hypertrophy was restrained by P-G3 treatment in both eWAT and iWAT (FIG. 2H,I), underlying their reduced depot sizes (FIG. 2G). Consistently, the expression of adipocyte markers in eWAT, including key adipogenic regulators (Cebpb, Pparg1, Pparg2, and Cebpa) and pan-adipocyte markers (Fabp4, Adipoq, Plin1, Cd36, and Lep) were dramatically suppressed (FIG. 2J and Extended Data FIG. 3a). This extent of repression of adipocyte genes is not typical in regular obesity-resistant models but similar to mice lacking adipose tissue, namely lipoatrophy^{12,13}. Lipoatrophy is associated with adipose tissue inflammation, insulin resistance, dyslipidemia, and liver steatosis. However, P-G3-treated mice showed comparable levels of glucose, insulin, free fatty acids (NEFA), and TG to control mice (Extended Data FIG. 3b-e). Neither did they develop hepatic steatosis but rather showed repressed expression of gluconeogenic (Foxo1 and G6pc) and lipogenic (Pklr and Scd1) genes with normal glycogen storage (Extended Data FIG. 3f,g), suggesting uncompromised or even improved liver metabolic health. Moreover, plasma alanine aminotransferase (ALT), the liver injury marker, was not increased in P-G3-treated mice (Extended Data FIG. 3h). Interestingly, macrophage marker F4/80 was increased in the eWAT of P-G3-treated mice but without inducing inflammatory genes Tnfa and 116 (Extended Data FIG. 3i,j). Instead, the anti-inflammatory M2 macrophage markers I16, Cd206, and Arg1 were up-regulated, further supporting the healthy remodeling of visceral fat by P-G3. Despite the similar inhibition of adipocyte hypertrophy in iWAT (FIG. 2I), its gene expression was in striking contrast to eWAT, with the largely normal expression of adipocyte genes (FIG. 2K). Therefore, the global repression of adipocyte genes in eWAT likely arises from the direct impact of P-G3, whereas the relatively normal adipocyte gene expression pattern in iWAT is secondary to the lean phenotype, coinciding with its biodistribution to visceral fat.

Indirect calorimetric analysis of energy homeostasis revealed that P-G3 treatment increased heat production and O₂ consumption (FIGS. 2L,M) without affecting locomotor activity, respiratory exchange ratio (RER), and food intake (Extended Data FIG. 3k-m). Furthermore, the obesity-associated metabolic dysfunctions, specifically glucose intolerance and insulin resistance, were alleviated (FIGS. 2N,O). The downstream target of insulin signaling—GSK3β phosphorylation was consistently increased in the eWAT of P-G3-treated mice (Extended Data FIG. 3a). Moreover, P-G3 treatment did not affect lipid absorption and homeostasis (Extended Data FIG. 3n), nor lipolysis (Extended Data FIG. 3o,p). Overall, P-G3 treatment increases energy expenditure to underlie its inhibition of obesity, which leads to improved metabolic consequences.

### P-G3 Induces Distinct Molecular Remodeling in Visceral and Subcutaneous Fat

HFD feeding causes hypertrophy, the enlargement of adipocytes to store more lipids. In understanding P-G3's anti-obesity effect, we found that P-G3 retained adipocytes in the smaller size in both eWAT and iWAT (FIGS. 2H and 2I), underlying their reduced depot sizes (FIG. 2G). P-G3 treatment dramatically suppressed the whole spectrum of adipocyte genes in eWAT, including adipogenic regulators (Cebpa, Cebpb, Pparg1, and Pparg2) and pan-adipocyte markers (Fabp4, Adipoq, Plin1, and Cd36) (FIG. 2J). This extent of repression of adipocyte genes is not typical in regular obesity-resistant models but similar to mice lacking adipose tissue, namely lipoatrophy. Lipoatrophy is associated with adipose tissue inflammation, insulin resistance, dyslipidemia, and liver steatosis. However, P-G3-treated mice showed healthier adipose tissue morphology, comparable levels of glucose, insulin, free fatty acids (NEFA), and TG when compared with control mice (Extended Data FIGS. 3b-3e). Additionally, their livers did not develop steatosis like lipoatrophy mice. Instead, they showed repressed expression of gluconeogenic (Foxo1 and G6pc) and lipogenic (Pklr and Scd1) genes (Extended Data FIGS. 3f and 3g), indicating improved liver metabolic health. Therefore, P-G3 inhibited visceral adiposity in a beneficial manner.

Underlying the decreased size of subcutaneous iWAT in P-G3-treated mice was the inhibited adipocyte hypertrophy (FIG. 2I), similar to eWAT. However, iWAT gene expression was in striking contrast to eWAT, with the largely normal expression of adipocyte genes (FIG. 2K). Despite the similar morphological changes in iWAT and eWAT, the global repression of adipocyte genes in eWAT likely arises from the direct impact of P-G3, whereas the relatively normal adipocyte gene expression pattern in iWAT is secondary to the lean phenotype. The distinct effects of P-G3 on iWAT and eWAT coincide with its biodistribution to visceral fat.

### P-G3 Impedes Adipocyte Hypertrophic Growth

The profound fat-remodeling function of P-G3 prompted us to test any direct effect of P-G3 on adipocyte development by employing a classic in vitro adipogenesis model—3T3-L1 cells, which can be differentiated into lipid-containing adipocytes through hormonal induction of adipogenic genes and lipid-synthetic genes¹⁴. Less lipid accumulation was observed in P-G3-treated cells; however, there were more lipid-positive cells but with smaller droplet sizes (FIG. 3A).

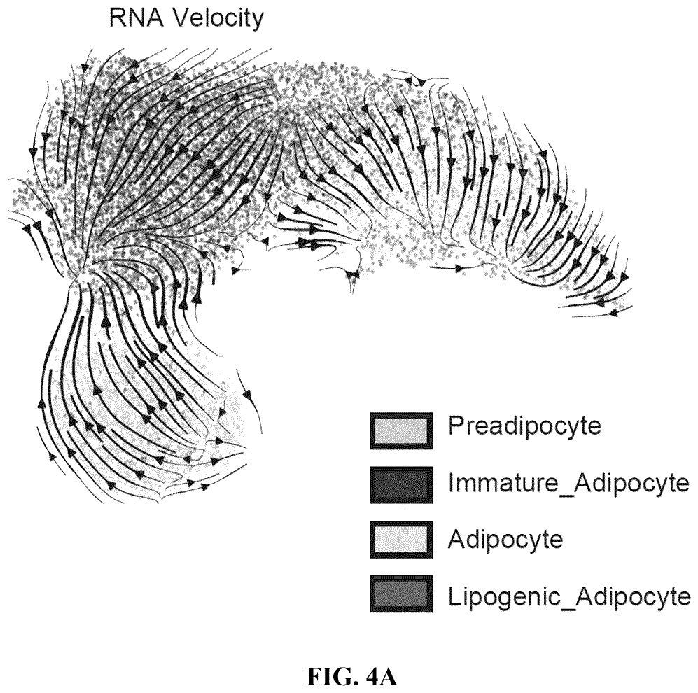

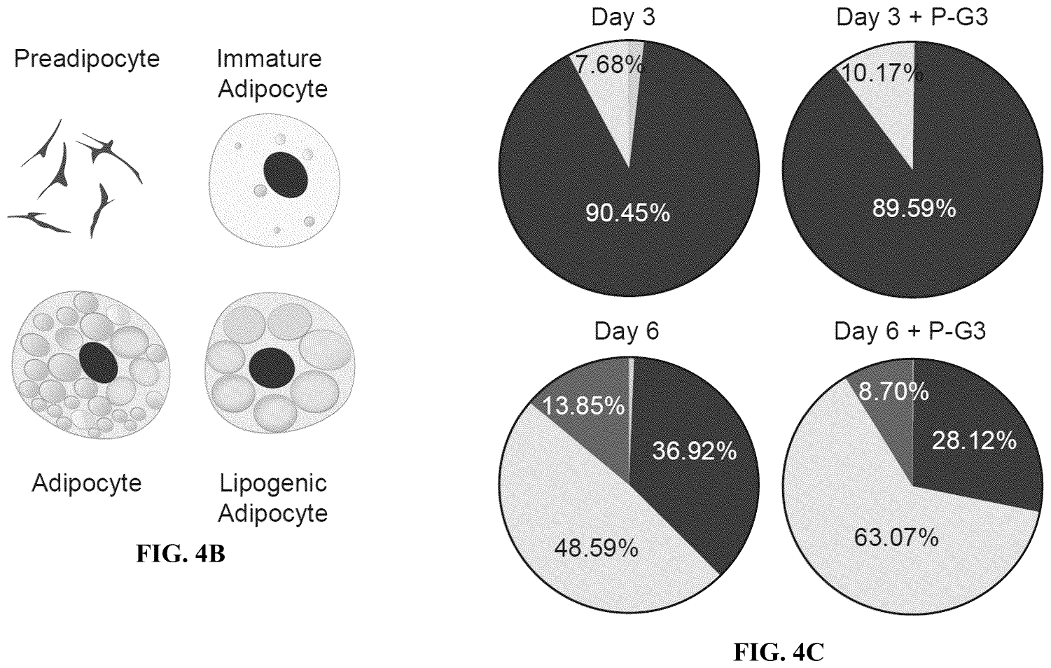

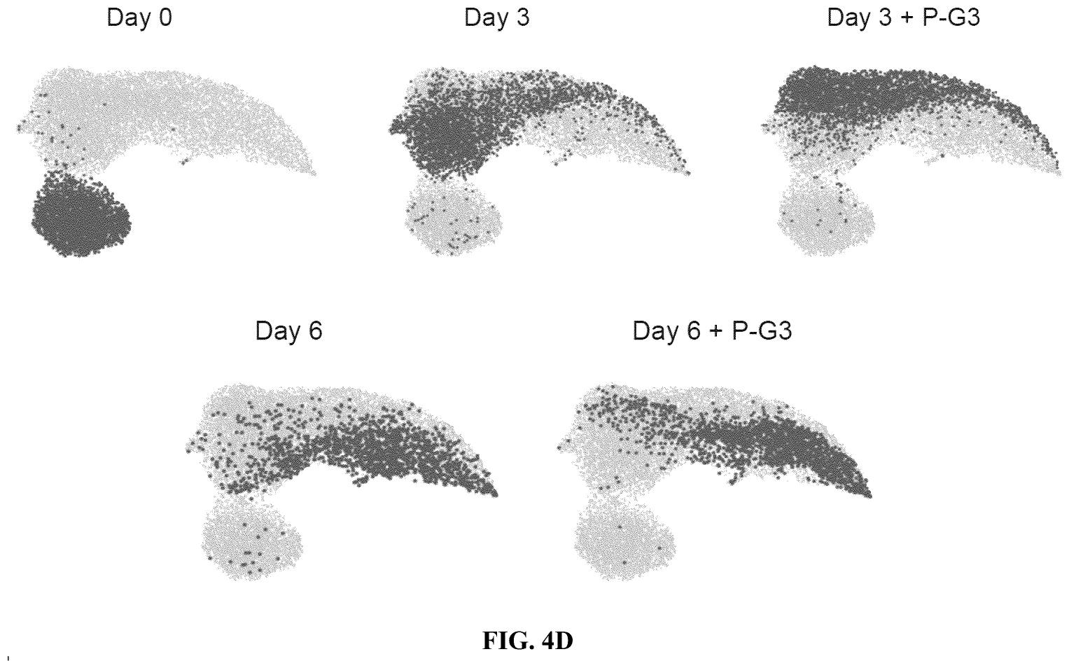

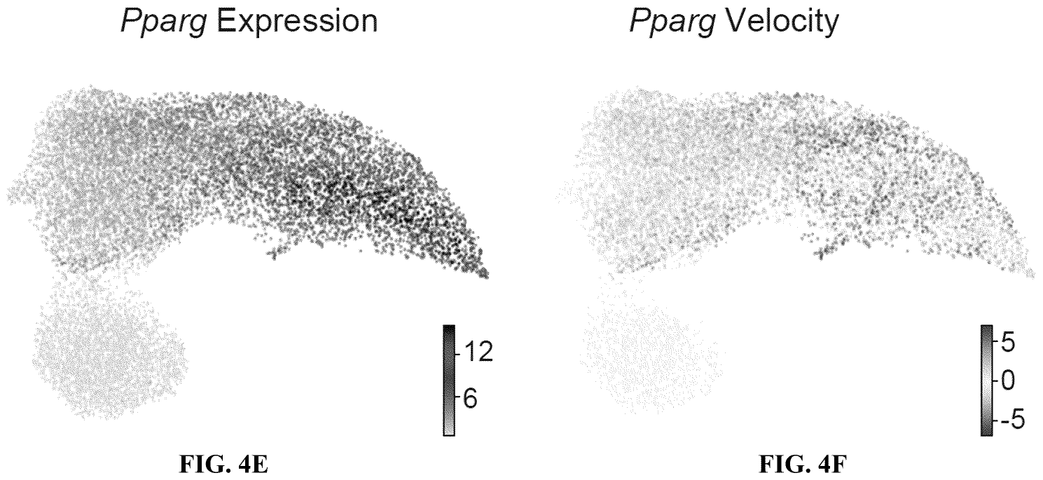

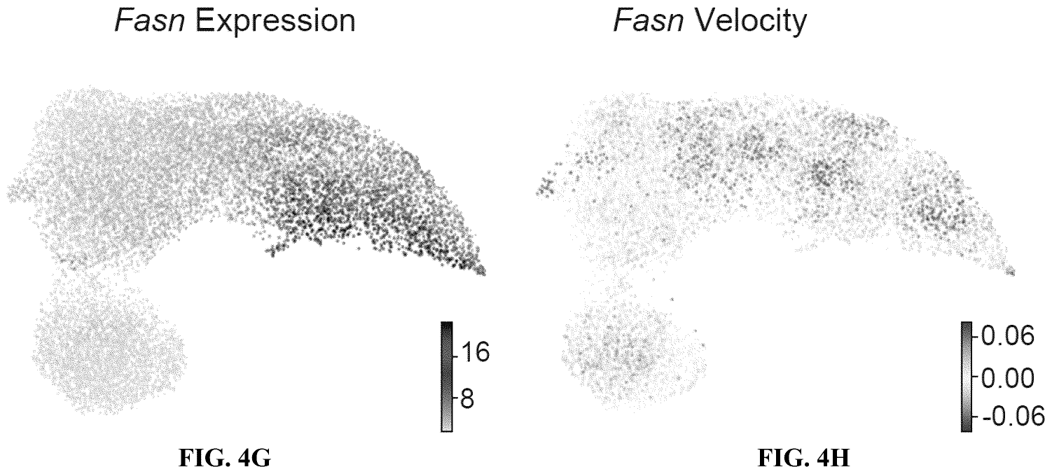

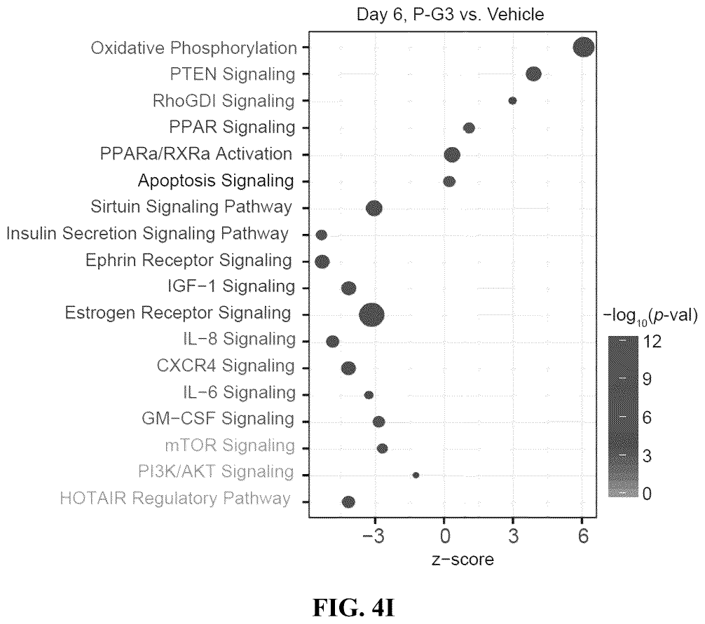

In a parallel C3H10T1/2 model, we observed faster lipid accumulation in early adipogenesis (Day 4) but similarly smaller lipid droplets after fully differentiated (since Day 6) (FIG. 3B and Extended Data FIG. 3a). Consistently, there was an overall earlier induction of key adipogenic factors by Day 4 of differentiation in P-G3-treated 3T3-L1 cells, including Cebpb, Pparg1, Pparg2, and Cebpa, and pan-adipocyte markers (Fabp4, Cfd, and Adipoq) (FIG. 3C); however, these mature adipocyte genes were all repressed in the following stage of hypertrophic growth (Extended Data FIG. 4b), likely owing to less lipid accumulation to sustain their maturation and phenocopying the overwhelmed repression in eWAT (FIG. 2J). This is particularly true for Plin1, a gene encoding a lipid droplet-coating protein Perilipin 1. It was normally induced during early adipogenesis but halted after Day 4 and significantly dropped in late differentiation by P-G3 treatment (Extended Data FIG. 4b), in direct correlation with the impaired lipid accumulation. In summary, P-G3 accelerates the adipocyte development program but inhibits hypertrophic growth.

The defining function of fat cells is to store lipids, which is essentially supported by the activation of genes for lipid synthesis. However, the inductions of the key lipogenic factors Fasn, Scd1, Srebf1, Acaca, Acacb, and glyceroneogenic gene Pck1 were blunted by P-G3 treatment during 3T3-L1 cell differentiation (FIG. 3D). This phenomenon was reproduced in human primary adipocytes (Extended Data FIG. 4c), with a more potent inhibition of lipogenic genes in the later stage (Extended Data FIG. 4d). Thus, P-G3 uncouples lipid synthesis from adipogenesis, two innately conjugated processes in adipocyte development, to create “dwarf” adipocytes, denoting normal adipocyte functions but deficient in lipid synthesis (FIG. 3E). This uncoupling also holds true in mature 3T3-L1 (FIG. 3F) and C3H10T1/2 (Extended Data FIG. 4e) adipocytes with transient P-G3 treatment. Moreover, the key TG synthetic genes Gpat3, Lipin1, and Dgat2 were also significantly repressed (FIG. 3F). This prevailing inhibition of lipid synthetic program by P-G3 was recapitulated in vivo in eWAT (FIG. 3G) but not in iWAT (Extended Data FIG. 4f). Importantly, ex vivo treating human omental fat with P-G3 led to a strong repression of key lipogenic genes in contrast to an upregulation of adipogenic genes CEBPB and FABP4 and blunted effects on PPARG1 and PPARG2 (FIG. 3H), indicating that P-G3's effects are translatable in human visceral fat.

Of note, acute 3-day P-G3 treatment primarily repressed lipid synthetic genes without affecting pan-adipocyte markers in eWAT (Extended Data FIGS. 4g, h), implying that the downregulation of adipocyte genes is secondary to mitigated lipid storage in chronic treatment. To further demonstrate an independent regulation of lipid synthesis from adipogenesis, we employed a PPARγ-overexpressing cell line to bypass the endogenous adipogenic cascade¹⁵. Again, P-G3 reduced their lipid droplet size (Extended Data FIG. 4i), reinforcing the uncoupling of lipid synthesis from adipogenesis. In addition, this uncoupling requires finetuning charge and structure since B-PEI, unlike P-G3, showed toxicity to adipocytes in vitro (Extended Data FIG. 4j).

### P-G3 Dulls Nutrient-Sensing Signals in Adipocyte Development

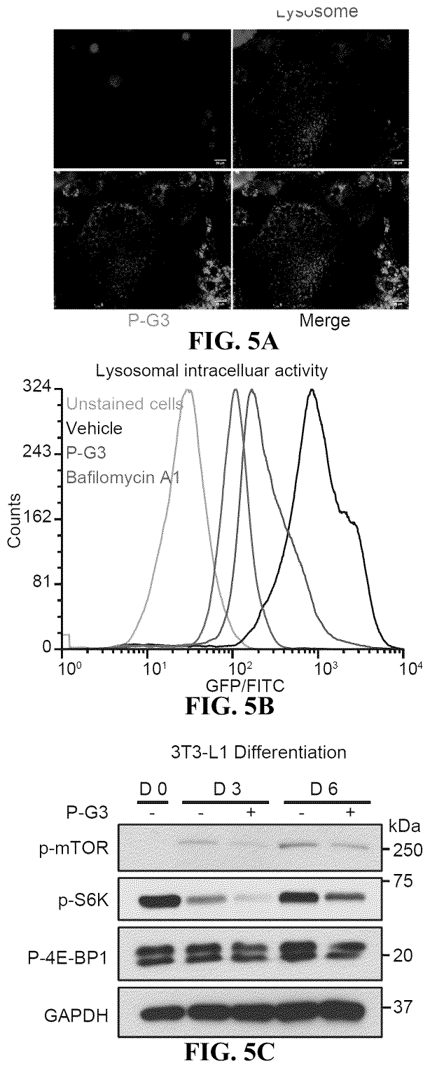

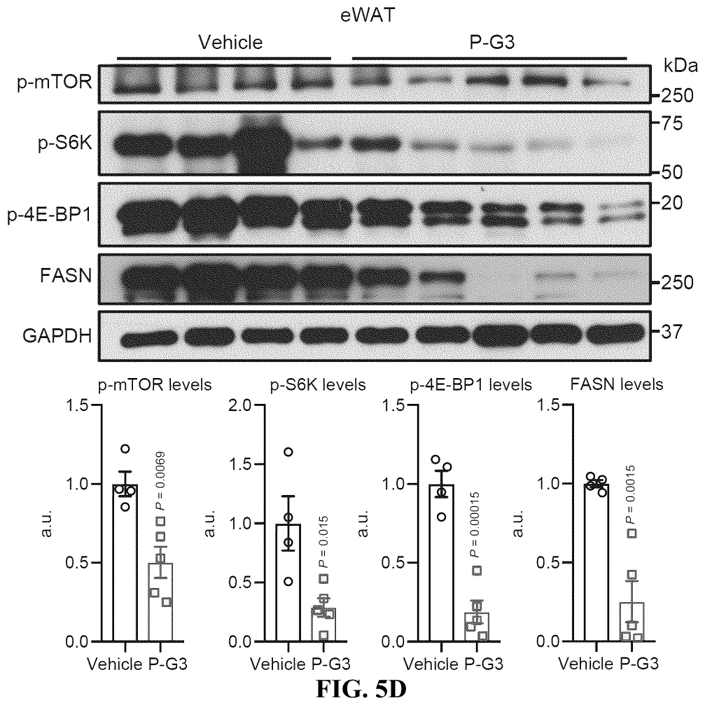

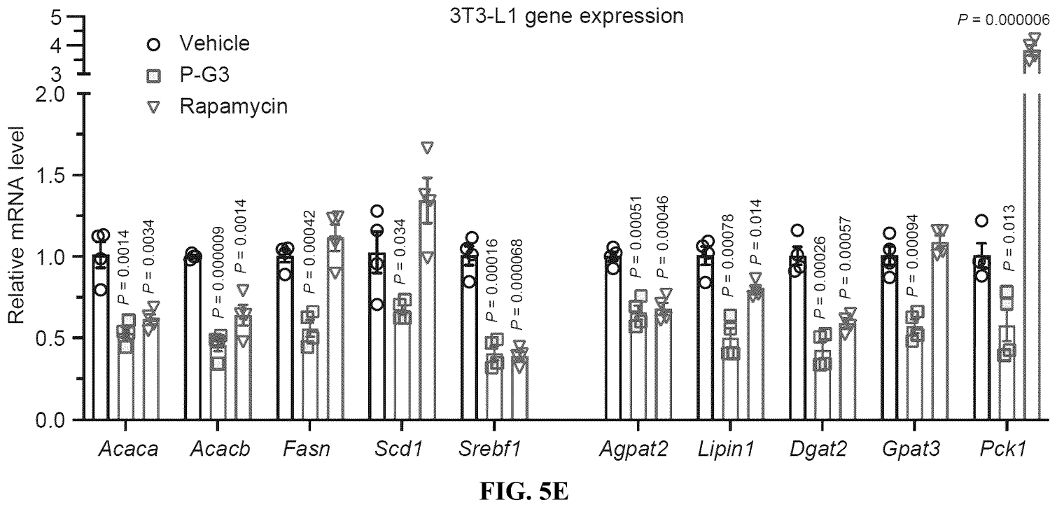

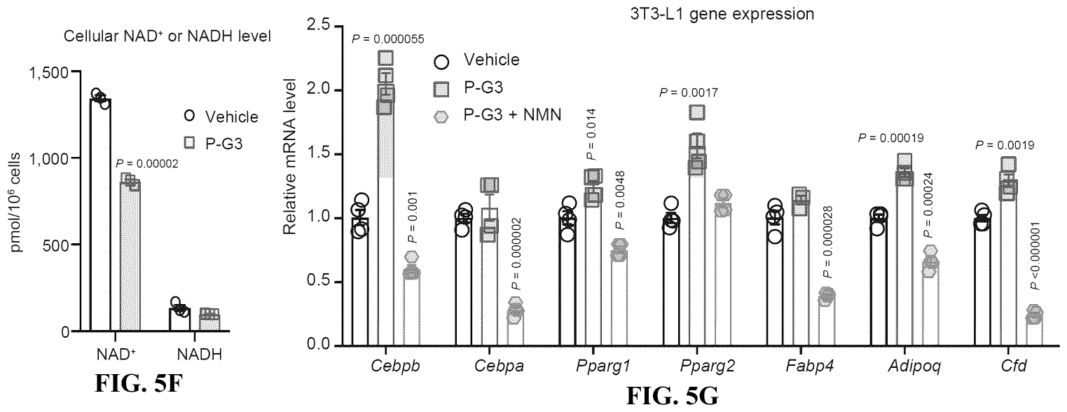

To understand P-G3's bifurcate regulation of adipocyte development, we performed scRNA-seq during 3T3-L1 cell differentiation. The heterogeneity and dynamics of adipogenesis were resolved at a single-cell level for the first time, clustered as preadipocytes, immature adipocytes, adipocytes, and lipogenic adipocytes (FIGS. 4A,B, and Extended Data FIG. 5a). Consistent with its pro-adipogenic function (FIG. 3C), P-G3 accelerated early adipogenesis at Day 3 of differentiation, indicated by a higher adipocyte proportion (10.17% vs. 7.68% in control) (FIGS. 4C,D, and Supplementary Table 1). However, despite their faster adipogenesis, P-G3-treated cells were reluctant to progress into the mature stage, with a population of 8.70% lipogenic cells on Day 6, compared to 13.85% in the control cells. The upregulation of adipocyte genes and downregulation of lipid-related genes by P-G3 treatment were ultimately resolved at the single-cell level (Extended Data FIG. 5b), buttressing the “dwarf” adipocyte phenotype.

Next, we identified driver genes in altered adipocyte development using RNA velocity and Regulon analyses (Extended Data FIG. 5c,d)¹⁶⁻¹⁸ Notably, the gene expression pattern was different from RNA velocities, the dynamic rates of mRNA synthesis, splicing, and degradation (FIGS. 4E-H). For example, the mRNA expression of Pparg, the master adipogenic factor, was high in mature adipocytes (FIG. 4E), but its unspliced/spliced mRNA velocity was stimulated in the premature adipocytes (FIG. 4F). Similar patterns were observed in the key lipogenic gene Fasn (FIGS. 4G,H) and other representative adipogenic genes (Extended Data FIG. 5e), suggesting that the active regulations of these genes are initiated prior to reaching their full expression. Furthermore, Qiagen ingenuity pathway analysis identified the altered pathways in lipogenesis and adipogenesis. P-G3 prompts oxidative phosphorylation, PPAR and PPARα/RXRα signaling but downregulates pathways in nutrient sensing, mTOR, nicotinamide adenine dinucleotide (NAD), Sirtuin signaling, and inflammation (FIG. 4I). Our study thus provides a framework of single-cell analysis assessing the cellular heterogeneity and changes upon P-G3 treatment.

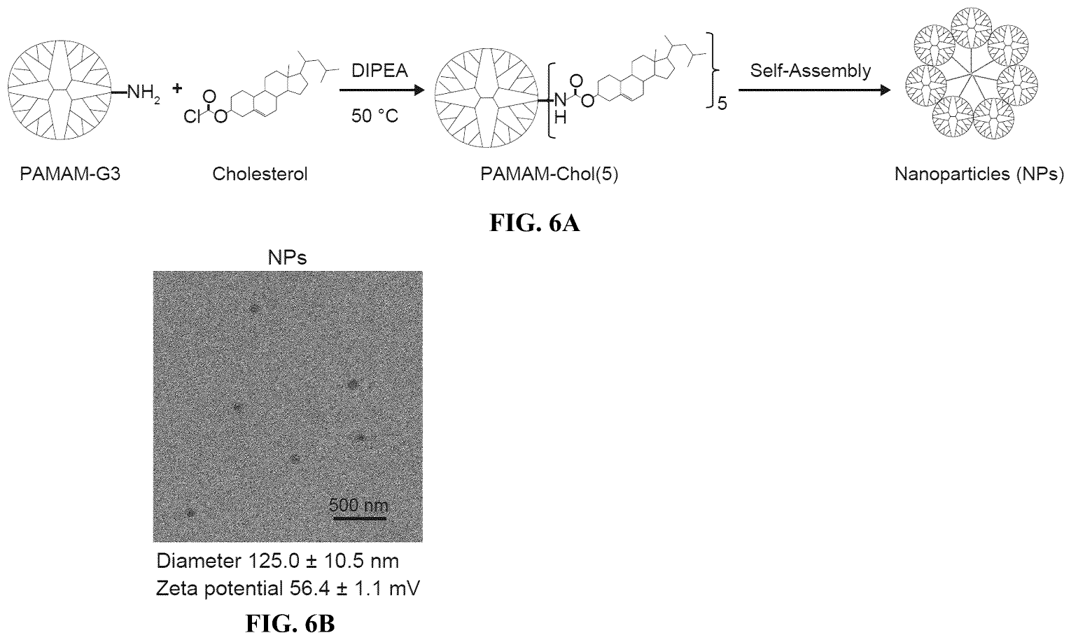

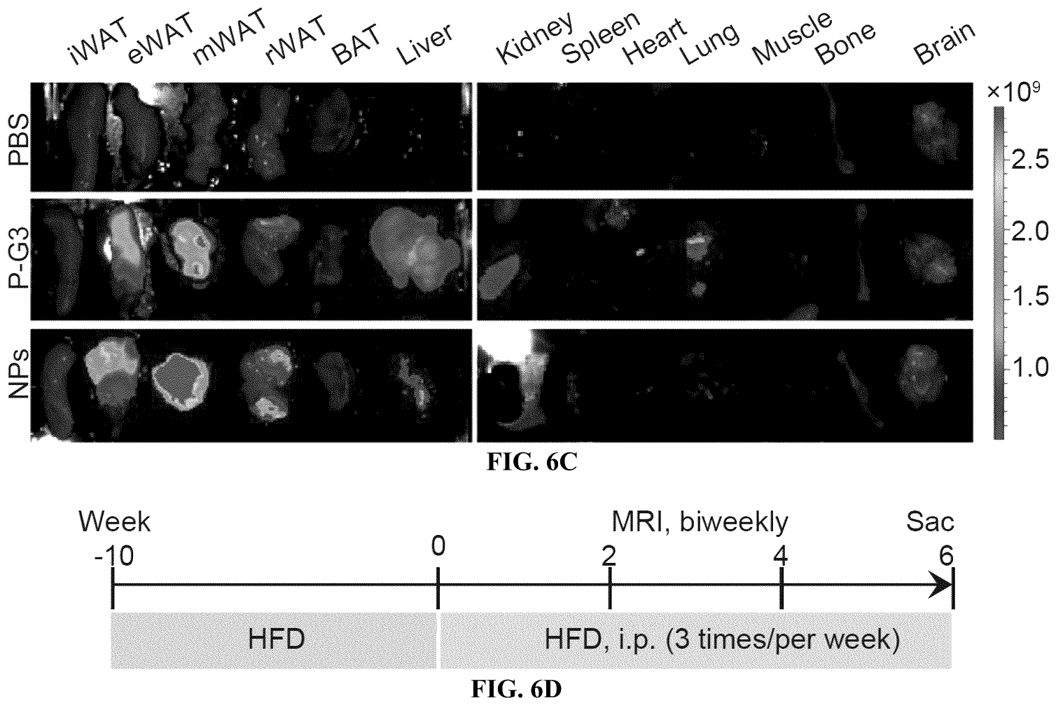

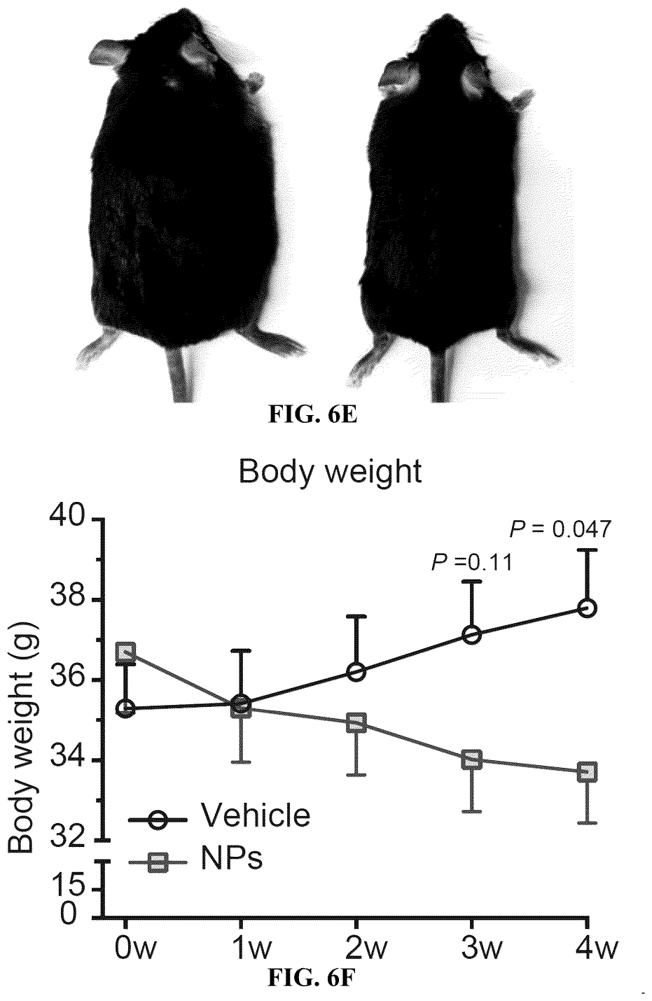

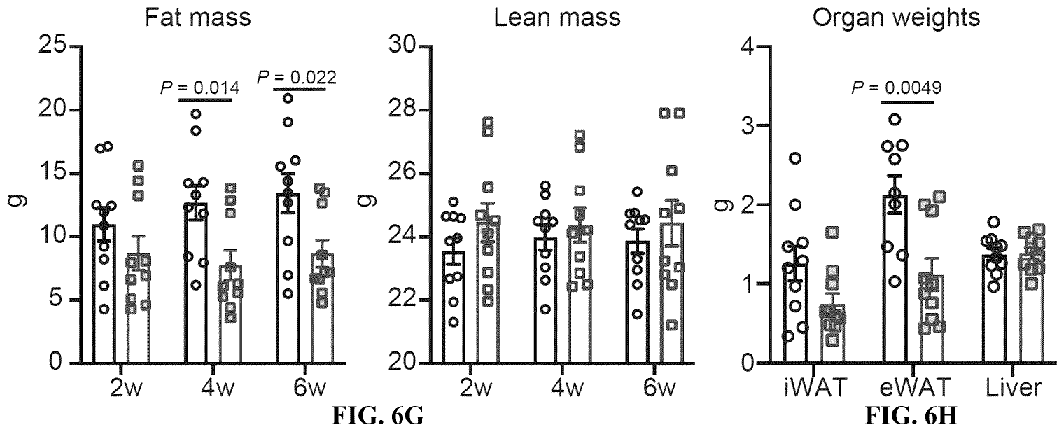

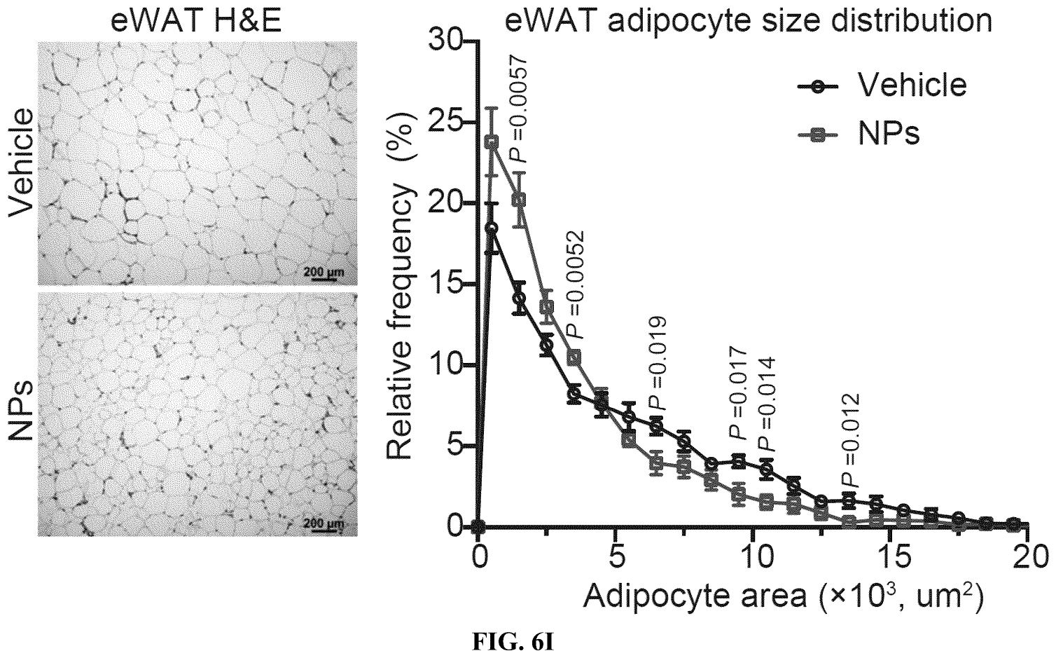

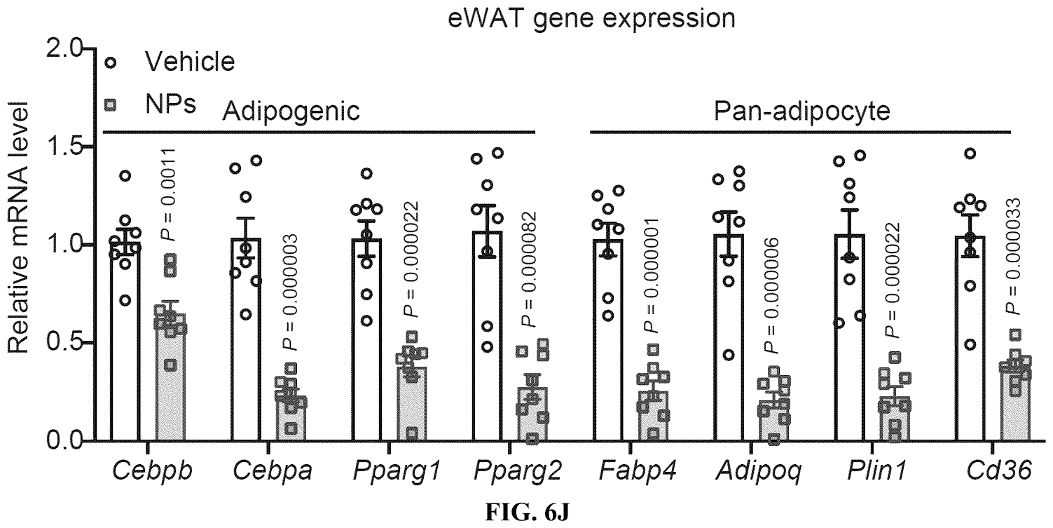

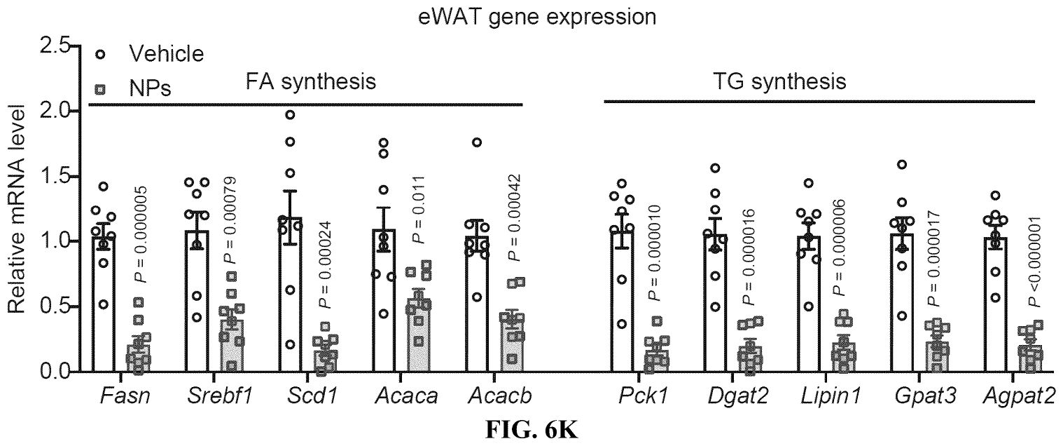

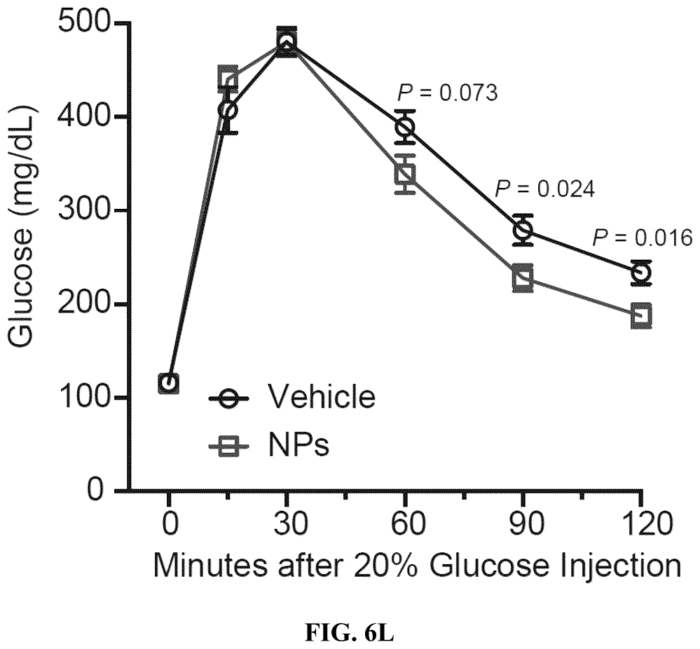

To determine the functioning size of P-G3 to affect adipocyte development, we conjugated P-G3 to carboxylate microbeads to attach to cell surface but block its entry into the cell; however, adipogenesis was impaired (Extended Data FIGS. 6a,b). It is possible that P-G3 changes the extracellular environment to affect adipocytes. We, therefore, incubated the P-G3 beads in the transwell to prevent their contact with adipocytes but observed minimal effect on adipocyte differentiation (Extended Data FIGS. 6a,b). Hence, the uncoupling effects of P-G3 on adipocytes require its entry into the cells. Cationic materials are known to enter cells mainly through endocytosis¹⁹⁻²¹. We detected colocalization of P-G3 with early endosomes in C3H10T1/2 adipocytes at 1 hour of treatment following the earlier colocalization with the plasma membrane at 15 minutes (Extended Data FIG. 6c). Over time, the accumulation of P-G3 was observed in lysosomes, while only weak colocalization was detected in the ER, mitochondria, or lipid droplets (FIG. 5A and Extended Data FIG. 6d). It is plausible that the cationic P-G3 may inhibit the acidification of lysosome to interfere with the latter's function. Indeed, P-G3 treatment in 3T3-L1 adipocytes decreased lysosomal activity ˜10-fold, mimicking the lysosome inhibitor bafilomycin A1 (FIG. 5B and Extended Data FIG. 6e). scRNA-seq analysis revealed the down-regulation of the mTOR pathway, which is critical for lipid synthesis in adipocytes²². mTOR localizes on lysosome, and its activity depends on the acidification of lysosome²³. We, therefore, asked whether P-G3 represses the mTOR pathway in adipocytes. P-G3 suppressed the mTOR activation as indicated by its decreased phosphorylation and that of downstream substrates S6K and 4E-BP1 during adipogenesis (FIG. 5C). The inhibition of mTOR signaling by P-G3 was potentiated in eWAT along with a strong inhibition of fatty acid synthase (FASN) (FIG. 5d). Moreover, treating 3T3-L1 (FIG. 5E) and C3H10T1/2 (Extended Data FIG. 6f) adipocytes with rapamycin, an mTOR inhibitor, largely mimicked P-G3's inhibition of the lipid synthetic genes (FIG. 5E). Of note, compared with rapamycin, the inhibition by P-G3 is more prevailing with additional repression of Fasn, Scd1, Pck1, and Gpat3 (FIG. 5E). These data demonstrate that P-G3 can impair lysosome function and inhibit mTOR activity in adipocytes, partially explaining the repression of lipogenesis.

Regarding the promotion of early adipogenesis by P-G3, scRNA-seq analyses revealed repression on the NAD signaling pathway and the Sirtuin signaling pathway (FIG. 4I), the latter of which is NAD⁺-dependent. NAD is a critical metabolite in regulating various cellular functions such as energy metabolism and differentiation²⁴. A precipitous decrease of NAD⁺ is required for adipocyte progenitors entering adipogenesis²⁵. Indeed, P-G3 efficiently decreased cellular NAD⁺ levels in preadipocytes, while there was no decrease in mature adipocytes (FIG. 5F and Extended Data FIG. 6g). We then reasoned that if P-G3 prompts early adipogenesis by decreasing NAD⁺ levels, this effect should be abolished by retaining NAD⁺. Supplementing NAD precursor nicotinamide mononucleotide (NMN) during the early adipogenesis stage to boost the intracellular NAD⁺ levels counteracted the pro-adipogenic effect of P-G3 (FIG. 5G). Therefore, P-G3 functions through a synergetic mechanism, likely involving repressed NAD and mTOR signaling, to dissociate lipogenesis from adipogenesis.

### Lipophilic P-G3 Nanoparticles Improve Visceral Fat Targeting

To further improve the visceral fat distribution of P-G3 and reduce the risk of off-target in other tissues, we covalently attached a lipophilic chain of 5 constitutive cholesterol molecules to P-G3 (FIG. 6A and Extended Data FIG. 7a). The resulting P-G3-Chol(5) could self-assemble in water to form spherical nanoparticles (NPs, size 125.0±10.5 nm) and maintain a cationic surface (zeta potential 56.4±1.1 mV) (FIG. 6B). P-G3-Chol(5) NPs showed the same endocytic uptake pattern by adipocytes as unmodified P-G3 (Extended Data FIG. 7b) and similarly promoted adipogenesis (Extended Data FIG. 7c). More importantly, the NPs displayed a comparable or slightly higher distribution to visceral fat depots as P-G3, but its distributions to the liver, kidney, and lung were significantly lower (FIG. 6C and Extended Data FIG. 7d), without affecting the penetration into visceral fat (Extended Data FIG. 7e).

Encouraged by the improved visceral fat specificity of NPs, we tested whether it can offer a therapeutic option in treating visceral obesity (FIG. 6D). In DIO mice with established obesity, 4-wk treatment resulted in a leaner phenotype (FIG. 6E) with a 15% decrease in body weight on continuous HFD feeding (FIG. 6F). The lean phenotype was exclusively attributed to a 45% reduction of fat mass as determined by EchoMRI (FIG. 6G). Consistently, eWAT depot size was reduced by ˜50%, with less reduction of iWAT (FIG. 6H). The adipocyte hypertrophy in obese eWAT was rectified by NPs treatment (FIG. 6I), which was supported by a prevalent suppression of adipogenic regulators, pan-adipocyte markers, and lipid biosynthesis genes in the existing mature adipocytes in eWAT (FIGS. 6J,K), accompanied by up-regulation of anti-inflammatory M2 macrophage markers (Extended Data FIG. 7f). In line with P-G3's inhibition of lipid synthesis but unaffected lipolysis, NP-treated mice regained less fat during refeeding rather than losing more fat mass during fasting (Extended Data FIG. 7g). As expected, NPs-treated mice showed improved glucose tolerance (FIG. 6L). Again, the mTOR signaling pathway was inhibited in NPs-treated eWAT (Extended Data FIG. 7h). Collectively, P-G3-Chol(5) NPs show promising therapeutic potential in treating visceral obesity in DIO mice.

### PG-3 For Delivery of Treating Agents

This technology may be used to provide targeted delivery of additional treating agents, along with the PG-3, to adipose tissue. Such treating agents may be used, for example, to treat obesity, diabetes, liver metabolic diseases, and aging. Liver metabolic diseases include, without limitation, Nonalcoholic fatty liver disease (NAFLD), nonalcoholic steatohepatitis (NASH), hereditary hemochromatosis, Alpha-I Antitrypsin Deficiency (AATD), and Wilson Disease. Suitable treating agents include, for example, agents that affect adipocytes, whether hydrophobic or hydrophilic; anti-inflammatory agents, including without limitation aspirin, ibuprofen, celecoxib, indomethacin, ordiclofenac, and other NSAIDs or other COX-2 inhibitors; antifibrotic agents, such as pirfenidone or Nintedanib; anti-senescence compounds, such as dasatinib, navitoclax, quercetin, or fisetin; or suitable nucleotides in any form, such as RNA, DNA, virus, etc.

### Methods

### Animal Studies

All mice were on a C57BL/6J background maintained in Columbia University animal facility with at 23±1° C. and a 12-hr light and dark cycle with ad libitum access to chow food (PicoLab Rodent 5053) and water. The HFD (high-fat diet) containing 60% fat was purchased from Research Diets (Cat #: D12492i). For the obesity prevention studies, male or female mice were fed HFD and i.p. injected with P-G3 (10 mg/kg·BW) in PBS twice weekly for the indicated times. In the treatment of lean mice, 6-wk-old male mice were fed chow diet and i.p. injected with P-G3 twice weekly for 6 weeks. For the obesity treatment study, male mice were induced obesity after 10 weeks' HFD feeding, and then i.p. injected NPs (10 mg/kg·BW) three times weekly for another 6 weeks. Body weight was monitored weekly, and body composition was determined by EchoMRI. After 16-hr fasting followed by 4-hr refeeding, mice were sacrificed by C02 euthanasia for tissue and plasma collection. Plasma insulin (Insulin ELISA, Mercodia), triglycerides (Thermo Scientific), and Non-esterified fatty acids (NEFA, Fujifilm Wako) were measured accordingly. To measure lipid absorption, lipid was extracted from fecal samples as described previously³⁷ and determined free fatty acid (FFA) content (NEFA, Fujifilm Wako). The Columbia University Animal Care and Utilization Committee approved all animal studies.

### Metabolic Phenotyping

For indirect calorimetric study, a separate cohort of male mice were subjected to the Comprehensive Lab Animal Monitoring System (CLAMS) (Columbus Instruments) after three P-G3 injections since the beginning of HFD feeding. For GTT, mice were fasted in a clean bedding cage for 16 hrs, then i.p. injected glucose (2 g/kg BW). Blood glucose was measured by using a Breeze2 glucometer (Bayer) at indicated time points. For ITT, mice were fasted for 4 hrs and i.p. injected with insulin (0.75 U/kg BW). For Lipid tolerance test, mice were fasted for 4 hrs and then given olive oil (200 μl/mice) by oral gavage. The blood samples were taken by tail vein bleeding at the indicated time points. Thereafter, serum lipid contents were measured accordingly. For lipolysis, mice were i.p. injected with isoproterenol (10 mg/kg. BW) and collected blood through tail vein bleeding, and serum NEFA (Fujifilm Wako) and glycerol (Sigma, F6428) levels were measured accordingly.

### Adipose Tissue ECM Isolation

WAT was decellularized following a previously described method with modifications³⁸. Briefly, tissue was placed in 50 ml centrifuge tube containing 0.02% trypsin-0.05% ethylenediaminetetraacetic acid (EDTA) solution with orbital shaking at 37° C. for 30 min, then incubated with a new trypsin-EDTA solution for another 30 min digestion. Next, tissue was sequentially incubated in the following solutions at room temperature (RT) with shaking: 3% Triton X-100 for 1 hr, 4% deoxycholic acid solution for 1 hr, and 4% ethanol and 0.1% peracetic acid for 2 hr. The tissue was rinsed in distilled deionized water (ddH₂O) between solution changes. Tissue was then washed at RT in phosphate-buffered saline (PBS) (pH 7.4) for 15 min three times, then in ddH₂O for 15 min three times, and in 100% n-propanol for 30 min twice. Lastly, tissue was washed four times with ddH₂O for 1 hr before being ready to use.

### Cy5-Labeled P-G3 Imaging

Ex vivo tissue distribution imaging: Mice were fed with HFD for 5 days to eliminate possible trace background signal from the chow diet and then collected tissues. The tissues and ECM from iWAT and eWAT were incubated in PBS with or without Cy5-labeled P-G3 (100 μg in 30 ml) for 45 min with shaking at 37° C. Afterward, the tissues were washed with PBS for 4 times and then subjected to imaging analysis using PerkinElmer IVIS Spectrum Optical Imaging System (Living Image 4.5.5 Software).

In vivo tissue distribution imaging: Chow-fed or HFD-fed mice were injected Cy5-labeled polymers or NPs (200 μg/mice) via intraperitoneal (i.p.) or intravenous (i.v.) routes, or locally into subcutaneous inguinal WAT. At the given time points, the mice were subjected to in vivo imagining by using PerkinElmer IVIS system (Living Image 4.5.5 Software). Mice were then sacrificed, and tissue signals were measured by using the same system.

### Cell Culture and Adipocyte Differentiation

3T3-L1 (ATCC CL-173) and C3H10T1/2 (CCL-226) cells were purchased from ATCC and cultured in high glucose DMEM supplemented with 10% calf serum (CS, Gemini Bio-Products 100-506) or FBS (heat-inactivated, Corning 35-011-CV), and 1× Pen Strep (Thermo Fisher). Cells were differentiated in the standard adipogenic cocktail after reaching confluence for 2 days. The cocktail contains DMEM, 10% FBS, 1 μM dexamethasone (DEX), 10 μg/ml insulin and 0.5 mM 3-isobutyl-1-methylxanthine (IBMX). 5 μM Rosiglitazone was used in the first two days to facilitate C3H10T1/2 cells' differentiation. After induction for two days, cells were maintained in a complete medium containing 2.5 μg/ml insulin until fully differentiated. P-G3 polymers or NPs (10 μg/ml) were added at indicated times, and cells were harvested during differentiation for analyses. Lipid droplets were visualized using Oil Red O staining or BODIPY staining. 3T3-L1 cells were treated with rapamycin (100 nM) from Day 4 to Day 6. C3H10T1/2 cells were treated with rapamycin (100 nM) from Day 4 to Day 9 for long-term rapamycin function analyses. 3T3-L1 cells were treated with NMN (20 mM) during Day 0 to Day 3 of differentiation and harvested on Day 4 for analyses.

Human pre-adipocytes were cultured and differentiated following a previously published protocol³⁹. Cells were differentiated in complete differentiation medium with or without P-G3 (10 μg/ml) from Day 0 to Day 7, and then switched to maintenance medium with or without P-G3 since day 7. Cells were harvested at Day 7 or Day 9 of differentiation for further gene expression analysis.

### Human Fat Explant Treatment

The protocol of collecting human fat biopsy has been reviewed and approved by Weill Cornell Medicine IRB (19-05020126). Fresh human omental adipose tissue was minced into small pieces (about 10 mg), washed with room-temperature PBS, and cultured in Medium 199 supplemented with 50 μg/ml gentamicin, 7 nM insulin and 25 nM DEX⁴⁰, at the same time treated with or without 10 μg/ml P-G3. Medium was replaced every 3 days and tissues were harvested on Day 8 of treatment for RNA analysis.

### Oil Red O Staining

Differentiated adipocytes were rinsed with PBS twice, then fixed in 4% formalin buffered solution for 30 min. Next, the fixed cells were washed with water twice, followed by covering with 60% isopropanol for 5 min, and then stained with freshly made and filtered 60% Oil red O isopropanol solution for 15 min. The cells were then washed with distilled water and imaged.

### Early Endosome Staining

C3H10T1/2 cells were plated on cover glass in 6-well plate (Corning, 22 mm×22 mm, No. 1) and differentiated using the protocol as described above. An early endosome marker (Cell Light Early Endosomes-GFP, BacMam 2.0, Thermo Fisher Scientific, 12 μl each well) was added into cell medium to stain early endosome overnight. After washing with PBS, Cy5-labeled P-G3 or Cy5-labeled NPs was added to the culture medium with a final concentration of 100 μg/ml. After 15 minutes or 1 hr incubation, Cy5-labeled P-G3 or Cy5-labeled NPs was removed, and then the cells were rinsed with PBS and fixed in 4% paraformaldehyde. Nuclei were stained with DAPI. The images were taken on a Nikon A1 confocal microscope.

### Cellular Organelle Staining

Mature 3T3-L1 cells were plated into 4-well Lab-Tek II Chambered cover glass (Thermo Fisher Scientific). Cy5-labeled P-G3 was added to the culture medium with a final concentration of 10 μg/ml. After 24 hr, cells were changed with a fresh medium containing 50 nM LysoTracker Red DND-99 (Thermo Fisher Scientific) or 100 nM MitoTracker™ Red CMXRos (Cell Signaling Technology) to stain lysosome or mitochondria, respectively, for 30 min. For endoplasmic reticulum (ER) staining, cells were rinsed with Hank's Balanced Salt Solution (HBSS) with calcium and magnesium, 1 μM ER-Tracker™ Red dye (Thermo Fisher Scientific) was added together with Hoechst 33243 for nucleus staining in live cells. For staining of lipid droplets, cells were fixed with 4% paraformaldehyde, washed with PBS, then incubated in 1 μM BODIPY 493/503 (Thermo Fisher Scientific) for 5 min followed by washing with PBS for 3 times. The images were taken on Zeiss Axio Observer 7.

### P-G3 Coating Carboxylate Microbeads

Excess P-G3 polymers were added to the carboxylate microbeads (Polybead® Carboxylate Microspheres 6.00 μm, Polyscience) in a microcentrifuge tube, vortexed briefly, and incubated with shaking overnight at 4° C. Microbeads were washed with cell culture-grade water, then spun down at 4° C., 10,000 g for 5 min. After washing three times, the precipitated microbeads were resuspended in cell culture grade water of the original volume, and the surface potential was measured with Malvern Zetasizer Nano ZS90. To quantify P-G3 contents on microbeads, Cy5 labeled P-G3 was used following the procedure above. P-G3 amount was determined according to the standard curve. 3T3-L1 or C3H10T1/2 cells were differentiated in 12-well transwell plate (Corning). 10 μg/ml P-G3 or microbeads containing an equal amount of P-G3 were added directly in the medium. In the transwell group, microbeads were separated from cells by an insert of 0.4 m pore size.

### NPs Synthesis and Characterization

15 μmol of P-G3 (Sigma-Aldrich) in 5 ml methanol (Thermo Fisher Scientific) was mixed with 75 mol of cholesterol chloroformate (Sigma-Aldrich) in 5 ml dichloromethane (Thermo Fisher Scientific), followed by the addition of 300 mol of N,N-Diisopropylethylamine (DIPEA, Sigma-Aldrich). The mixture was then stirred at 50° C. for 3 hr, and dialyzed in ultrapure water for 72 hr to generate P-G3-Chol(5). To fabricate P-G3-Chol(5) nanoparticles, 1 mg of P-G3-Chol(5) was dissolved in 200 μL of chloroform (Thermo Fisher Scientific), followed by the addition of 1 ml H₂O and sonication for 2 min. Finally, 5 ml of H₂O was added into the mixture, and the excess solvent was removed by using a rotary evaporator to generate P-G3-Chol(5) NPs. The structure of the resulting NPs was characterized by a Bruker Avance III 400 NMR instrument. The morphology was imaged using a Titan Themis 200 TEM. The hydrodynamic diameter and zeta potential of the nanoparticles were measured by a Malvern Nano ZS90 Zetasizer. The absorbance was measured using a Denovix DS-11+ Spectrophotometer.

### NAD⁺ Cellular Concentration

Preadipocytes or adipocytes were treated with P-G3 (10 μg/ml) or PBS for 14 hr, and the cellular NAD⁺/NADH levels were determined by the NAD⁺/NADH Quantitation Colorimetric Kits (Biovision K337) following the manufacturer's instructions.

### Gene Expression Analysis

Total RNA from tissues or cells was extracted by using the TriZol reagent (Thermo Fisher) in combination with an RNA isolation kit from Macherey-Nagel. 1 μg RNA was used for reverse transcription to synthesize cDNA by using High-Capacity cDNA Reverse Transcription kit (Applied Biosystems). Bio-Rad CFX96 Real-Time PCR system was used to perform quantitative real-time PCR (qPCR) with goTaq qPCR Master Mix (Promega). The relative gene expression was calculated by using the ΔΔCt method with Cyclophilin A or Rpl23 as the reference gene. Primer sequences are available in Supplementary Tables 4-5.

scRNA-seq

3T3-L1 cells were cultured and differentiated as described above. P-G3 (10 μg/ml) or PBS was added to the cells since Day 0 of differentiation. Cells were collected at differentiation Day 0, 3, and 6 as preadipocytes, early adipogenesis, and mature adipocytes, respectively (Supplementary Table 1). At collection, cells were gently dissociated into single cells with trypsin enzymatic digestion. Cell viability was confirmed over 85% by trypan blue exclusion before subjecting for scRNA-seq using 10× Genomics Chromium technology. The resulting single-cell 3′-end cDNA libraries were sequenced on Illumina® NovaSeq™ 6000 Sequencing System (2×100 bp pair-end) at the Single Cell Analysis Core of the Columbia Genome Center. 10× genomics' Cell ranger pipeline v3.1.0 with mouse reference transcriptome GRCm38 was used to process the data. Details of sample information for single cell RNA sequencing were listed in Supplementary Table 2. In samples containing mature adipocytes (LQ004 and LQ005), the cell suspension media also contained lipid droplets. Lipid droplets were coincidently barcoded as well. This was the reason for the high expected number from the sequencer with low mean reads in LQ004 and LQ005. We excluded the lipid droplets noise in sequencing data by only keeping the cells with gene numbers (2,500 to 9,000 genes) and mitochondria gene percentage less than 20% for downstream analysis. Details of software and algorithms could be found in Supplementary Table 3.

### RNA Velocity and Regulatory Network Analysis

Preprocessing of the single-cell analysis for normalization and unsupervised clustering was performed with Scanpy (v1.7.1) in Python (v3.6). We annotated the cell types based on known adipogenesis and lipogenesis markers. We classified the subgroup of the mature adipocytes as lipogenic adipocytes when a high expression of lipid accumulation genes was observed. Overall, four cell types were observed in our dataset: preadipocytes, immature adipocytes, adipocytes, and lipogenic adipocytes (FIG. 4B, Extended Data FIG. 5a, and Supplementary Table 1). Next, we computed the RNA velocities (rate of splicing and degradation) based on spliced and unspliced mRNA dynamics of every single cell using scVelo (v0.2.1) and Velocyto (v0.17.16) (FIGS. 4E-H, and Extended Data FIGS. 5d, e). Regulatory networks were identified by co-expression pattern of transcription factor and its downstream effector genes by SCENIC (v1.2.2) (Extended Data FIG. 5c). The methods for recovering RNA velocity and regulons are insensitive to data normalization. We used raw sequencing data and cell-type annotations as inputs to perform the above analysis.

### Altered Gene Pathway Analysis

We used Qiagen Ingenuity Pathway analysis (IPA) software to calculate the statistical significance in FIG. 4I. In brief, IPA software used Fisher's Exact Test to calculate the overlap of our dataset with reference database (i.e. sets of molecules in Canonical Pathways). The p-value measures the likelihood of an observed association between our experimental dataset and a specific canonical pathway due to random chance. The corresponding pathway is significantly associated with the experiment dataset when the p-value is small (i.e., p-value<0.05).

### Lysosomal Intracellular Activity Analysis

The lysosomal activity was determined by using the BioVision lysosomal intracellular activity assay kit (Catalog #: k448-50). In brief, C3H10T1/2 cells were pre-treated with or without P-G3 from differentiation Day 5 to Day 7. Bafilomycin A1 was used as a lysosome inhibitor control. The cells were then incubated in a cell medium supplemented with 0.5% FBS and self-quenched substrate for 2 hr, followed by the addition of assay buffer to terminate the experiment. The cells were then dissociated and subjected to flow cytometry analysis (488 nm excitation laser). The gating strategy were shown in Extended data FIG. 6e. Flow cytometry data were analyzed by FCS Express (7.14.0020) software.

### Western Blotting

Cells or tissues were lysed with total protein lysis buffer supplemented with protease and phosphatase inhibitor cocktail. Protein supernatants were subjected to protein quantification using Pierce BCA Kit and 10% gel SDS-PAGE separation. Antibodies used in this study are: p-mTOR (CST #2971), p-S6K (CST #9205), p-4E-BP1 (CST #2855), p-AKT (Ser 473, CST #9271), p-AKT (T308, CST #13038), p-GSK3b (Ser 9, CST #9322), FASN (CST #3180), ADIPSIN (R&D Systems, #AF5430), ADIPONECTIN (Thermo Fisher, #PA1-054), CEBPA (Santa cruz, sc-61), HSP90 (Proteintech, #13171-1-AP) and GAPDH (Proteintech #HRP-60004). The dilution of antibodies were based on the recommendation on the manufacturer's website.

### Histological Assessments

After dissection, eWAT, iWAT, and liver were immediately fixed in 10% formalin buffered solution. After fixation and dehydration, tissues were embedded into paraffin, stained with antibodies against F4/80 (CST, #70076), Hematoxylin and Eosin (H&E) or Periodic Acid-Schiff (PAS), and photographed under microscope (Olympus IX71). For immunohistochemistry, eWAT, iWAT, and liver were immediately fixed in 4% paraformaldehyde at 4° C. for overnight, and then dehydrated in 30% sucrose at room temperature for overnight, then embedded into OCT medium (Sakura Tissue-Tek® O.C.T. Compound 4583). After mounting in 5 m slides, the frozen sections were incubated with antibody Caveolin-1 (D46G3) (CST, #3267, 1:250 dilution) and then anti-rabbit Alexa 488 antibody (Thermo Fisher Scientific, #A27034, 1:1000 dilution), and further imaged by confocal microscopy. The images were processed using ImageJ 2.1.0 (National Institutes of Health) software.

### Statistical Analysis

The significance between groups was evaluated by using two-tailed Student's t-test. The difference between groups is statistically significant if the p value is less than 0.05. In this study, all data were represented as mean±s.e.m. Prism 9.3.1 (GraphPad) was used for analysis.

### Data Availability

The single-cell RNA-seq data are available in the Gene Expression Omnibus (GEO) database under accession number GSE209819. Sample information and sequencing statistics are described in Supplementary Tables 1-2. All the remaining data will be available from the authors upon reasonable request.

### SUMMARY

Targeting and reducing abdominal fat using polycations Treatment with polycations was shown to selectively target abdominal fat in mice owing to the negatively charged extracellular matrix in adipose tissue. The polycations can inhibit the storage of lipids in fat cells causing them to shrink, leading to improved metabolic health in obese mice.

### The Problem

The location, function, molecular profile, and intervenability of fat can vary. Visceral fat (that is, fat stored in the abdominal cavity) is particularly resistant to intervention because of its metabolic character and the fact that it is hard to access and operate on, it is also more harmful than fat that accumulates in other regions, such as underneath the skin¹. The accumulation of visceral fat increases the risk of various comorbidities such as type 2 diabetes mellitus, cardiovascular diseases, fatty liver disease and chronic inflammation¹. However, despite these associated risks no specific treatment for visceral fat has yet been developed.

### The Solution

Fat cells are immersed in an extracellular matrix composed of collagen and glycosaminoglycans, the latter of which are some of the most negatively charged biomacromolecules in the body. This negatively charged matrix may thus provide a way to attract and transport positively charged molecules. Cationic nanomaterials are nano-scale complexes that carry multiple positive charges. They have been widely tested as nucleotide cargo carriers or scavengers for negatively charged pathogens in treating inflammatory diseases²³. Therefore, cationic nanomaterials may build up within fat tissues given the negatively charged extracellular matrix. This enrichment could be used to tackle the challenge of targeting specific fat depots within the body.

To investigate this possibility, we injected a polyamidoamine generation 3 (P-G3) dendrimer, which is a polycation carrying 32 positive charges on its surface, into the abdomen of obese mice and found that the P-G3 preferentially targeted visceral fat over fat depots in other regions of the body (FIG. 1A). We found that treatment with P-G3 reduced the mass of visceral fat, leading to improved metabolic health, such as better glucose metabolism, higher energy expenditure, and improved liver function. This unexpected reduction in visceral fat is associated with a depletion in the ability of fat cells to store lipids while maintaining the development and identity of the fat cells, leading to the production of ‘dwarf’ adipocytes. These dwarf adipocytes are ideal because they function like normal adipocytes but are smaller in size and are resistant to the growth of metabolically unhealthy hypertrophic adipocytes (FIG. 1B).

It is likely that P-G3 functions by disturbing the nutrient-sensing pathways to shut down the lipid synthesis and storage programme. Therefore, a positive charge is not only the key to targeting visceral fat but also confers an intriguing mechanism to reduce fat. The ability of P-G3 to target fat over non-adipose tissue can be further improved by adding a cholesterol tail to form lipophilic nanoparticles.

### Applications

This discovery that positive charge is the key to targeting fat will open up avenues for cationized obesity treatment that can safely and effectively manipulate specific fat depots. Targeting specific fat depots with P-G3 requires the desired region to be directly exposed to P-G3, for example through abdominal injections for visceral fat. This characteristic can be leveraged to treat subcutaneous fat using local injections. Given the carrier capacity of cationic nanomaterials, they can deliver therapeutic agents in the form of small compounds, nucleotides, and proteins to a given fat depot⁴. Fat reduction could thus be increased by combining the effect of the cationic nanomaterial with the increased local drug concentration. Safety is a primary consideration when developing obesity intervention strategies, and cationic local delivery can avoid adverse effects arising from off-targeting in non-adipose tissues.

The stronger the positive charge, the more the nanoparticle will preferentially target fat; however, stronger positive charge also increases the toxicity of the nanoparticle. The window for the optimal efficacy to toxicity ratio should be carefully evaluated in terms of charge density, this ratio is likely to vary depending on the location of a specific fat depot. In this study, we injected the P-G3 into the abdomen to target visceral fat; however, less invasive and more convenient delivery routes are desirable, such as oral administration, sublingual or buccal administration, parenteral administration, transdermal administration, via inhalation or intranasally, vaginally, rectally, and intramuscularly. Parenterally may be by epifascial, intracapsular, intracranial, intracutaneous, intrathecal, intramuscular, intraorbital, intraperitoneal, intraspinal, intrasternal, intravascular, intravenous, parenchymatous, subcutaneous or sublingual injection, or by way of catheter.

### CONCLUSIONS

Polycation P-G3 is selectively distributed to visceral fat via the i.p. delivery route to inhibit visceral adiposity and prevent obesity by uncoupling lipid synthesis from adipocyte development, creating “dwarf” adipocytes. Engineering P-G3 into lipophilic NPs further improves its visceral fat-selective biodistribution and shows therapeutic potential to treat obesity.

More importantly, P-G3 and its NPs derivative overcome the critical barrier to tackling visceral obesity. Unlike subcutaneous fat with multiple available approaches²⁶, there is no option for treating visceral adiposity except surgical removal developed in rodents and baboons, regardless of the risk and complexity^{4,27}. Therefore, the present methodology presents a revolutionary cationic strategy for treating obesity, distinct from the existing anti-obesity interventions.

P-G3 renders adipocytes smaller, either from de novo adipocyte formation or in mature adipocytes. In contrast to hypertrophic ones, smaller adipocytes are usually metabolically healthier²⁸⁻³⁰, underlying the metabolic improvements by P-G3 treatment. These “dwarf” adipocytes arise from prevalent and selective inhibition of lipid synthetic genes. Hence, lipid storage does not necessarily coincide with adipocyte phenotypic development, and P-G3 can uncouple them. The divergent regulation of adipocytes by P-G3 differs from conventional adipocyte manipulations, such as adipocyte delipidation induced by nutrient deprivation or inflammatory reagents to cause the repression of pan-adipocyte genes and loss of adipocyte identity³¹⁻³⁴ or adipocyte browning involving activation of the thermogenic program without shutting down lipid synthesis. This unique effect of P-G3 is likely mediated through repressing mTOR and NAD⁺ signaling, two pivotal metabolic sensing nexuses. Hyperactivation of mTOR has been reported in obesity and T2DM³⁵, signifying the importance of maintaining appropriate mTOR activity. P-G3 may offer an option to fine-tune mTOR activity to improve metabolic disease management. It is also plausible that P-G3 would target multiple pathways to account for the metabolic benefits. For example, leptin was markedly repressed in the eWAT by P-G3 treatment (FIG. 2J). It is a key adipokine to regulate food intake and energy expenditure through functioning in the brain³⁶, raising the possibility that P-G3 may influence the adipose tissue-brain communication to tilt the whole-body energy balance toward expenditure.

Cationic charge correlates with polycation's efficacy but also with toxicity. B-PEI has relatively higher charge density and better visceral fat targeting efficiency, while its toxicity is obvious in vitro. P-G3 is likely at the sweet spot between efficacy and safety, which could be further optimized by cholesterol modification. Moreover, leveraging polycations' carrier capacity in combination with the visceral fat-specific targeting property demonstrated here, it is highly feasible to encapsulate fat-manipulating agents into P-G3 NPs to specifically deliver them into visceral fat for additive anti-obesity benefit but reduced off-target effects. Collectively, our study presents a strategy to target visceral adiposity, and cationic nanomaterials useful for treating metabolic diseases. These methods may also be used to deliver additional treating agents for treating metabolic diseases and other conditions, as discussed above.

### REFERENCES

L. Danaei, G. et al. The preventable causes of death in the United States: comparative risk assessment of dietary, lifestyle, and metabolic risk factors. PLoS Med. 6, e1000058 (2009).
2. Verboven, K. et al. Abdominal subcutaneous and visceral adipocyte size, lipolysis and inflammation relate to insulin resistance in male obese humans. Sci. Rep. 8, 4677 (2018).
3. Kajimura, S., Spiegelman, B. M. & Seale, P. Brown and beige fat: physiological roles beyond heat generation. Cell Metlab. 22, 546-559 (2015).
4. Huffman, D. M. & Barzilai, N. Role of visceral adipose tissue in aging. Biochim. Biophys. Acta 1790, 1117-1123 (2009).
5. Svenson, S. & Tomalia, D. A. Dendrimers in biomedical applications—reflections on the field. Adv. Drug Deliv. Rev. 57, 2106-2129 (2005).
6. Lee, J. et al. Nucleic acid-binding polymers as anti-inflammatory agents. Proc. Natl Acad. Sci. USA 108, 14055-14060 (2011).
7. Lee, J, et al. Nucleic acid scavenging microfiber mesh inhibits trauma-induced inflammation and thrombosis. Biomaterials 120, 94-102 (2017).
8. Pisetsky, D. S., Lee, J., Leong, K. W. & Sullenger, B. A, Nucleic acid-binding polymers as anti-inflammatory agents: reducing the danger of nuclear attack. Expert Rev. Clin. Immunol. 8, 1-3 (2012).
9. Nariman, F. C. & Wang, P. Adipocyte extracellular matrix composition, dynamics and role in obesity. Cell. Mol. Life Sci. 67, 1277-1292 (2010).
10. Puri, S., Coulson-Thomas, Y. M., Gesteira, T. F. & Coulson-Thomas, V. J. Distribution and function of glycosaminoglycans and proteoglycans in the development, homeostasis and pathology of the ocular surface. Front Cell Dev. Biol. 8, 731 (2020).
11. Esfand, R. & Tomalia, D. A. Poly(amidoamine) (PAMAM) dendrimers: from biomimicry to drug delivery and biomedical applications. Drug Disco. Today 6, 427-436 (2001).
12. Pajvani, U. B. et al. Fat apoptosis through targeted activation of caspase 8: a new mouse model of inducible and reversible lipoatrophy. Nat. Med. 11, 797-803 (2005).
13. Wang, F., Mullican, S. E., DiSpirito, J. R., Peed, L. C. & Lazar, M. A. Lipoatrophy and severe metabolic disturbance in mice with fat-specific deletion of PPARγ. Proc. Natl Acad. Sci. USA 110, 18656-18661 (2013).
14. Farmer, S. R. Transcriptional control of adipocyte formation. Cell Metab. 4, 263-273 (2006).
15. Li, D. et al. Distinct functions of PPARγ isoforms in regulating adipocyte plasticity, Biochem. Biophys. Res. Commun. 481, 132-138 (2016).
16. La Manno, G. et al. RNA velocity of single cells. Nature 560, 494-498 (2018).
17. Bergen, V., Lange, M., Peidli, S., Wolf, F. A. & Theis, F. J. Generalizing RNA velocity to transient cell states through dynamical modeling. Nat. Biotechnol. 38, 1408-1414 (2020).
18. Albar, S. et al. SCENIC: single-cell regulatory network inference and clustering. Nat. Methods 14, 1083-1086 (2017).
19. H-wang, M. E., Keswani, R. K. & Pack, 1). W. Dependence of PEI and PAMAM gene delivery on clathrin- and caveolin-dependent trafficking pathways. Pharm. Res. 32, 2051-2059 (2015),
20. Fox, L. J., Richardson, R. M. & Briscoe, W. H. PAMAM dendrimer-cell membrane interactions. Adv. Colloid Interface Sci. 257, 1-18 (2018),
21. Kitchens, K. M., Foraker, A. B., Kolhatkar, R. B., Swaan, P. W. & Chandehari, H. Endocytosis and interaction of poly (amidoamine) dendrimers with Caco-2 cells. Pharm. Res. 24, 2138-2145 (2007).
22. Saxton, R. A. & Sabatini. D. M. mTOR signaling in growth, metabolism, and disease. Cell 168, 960-976 (2017).
23. Chung, C. Y. et al. Covalent targeting of the vacuolar H(+)-ATPase activates autophagy via mTORC1 inhibition. Nat. Chem. Biol. 15, 776-785 (2019).
24. Rajman, L., Chwalek, K. & Sinclair, D. A. Therapeutic potential of NAD-boosting molecules: the in vivo evidence. Cell Metab. 27, 529-547 (2018).
25. Ryu, K. W. et al. Metabolic regulation of transcription through compartmentalized NAD(+) biosynthesis. Science 360, eaan5780 (2018).
26. Zhang, Y, et al. Locally induced adipose tissue browning by microneedle patch for obesity treatment. ACS Nano 11, 9223-9230 (2017).
27. Andrew. M. S. et al. Mesenteric visceral lipectomy using tissue liquefaction technology reverses insulin resistance and causes weight loss in baboons. Surg. Obes. Relat. Dis. 14, 833-841 (2018).
28. Ghaben, A. L. & Scherer, P. E. Adipogenesis and metabolic health. Nat. Rev. Mol. Cell Biol. 20, 242-258 (2019).
29. Tandon, P., Wafer, R. & Minchin, J. E. N. Adipose morphology and metabolic disease, J. Exp. Biol. 221, jeb164970 (2018).
30. Acosta, J. R. et al. Increased fat cell size: a major phenotype of subcutaneous white adipose tissue in non-obese individuals with type 2 diabetes. Diabetologia 59, 560-570 (2016).
31. Brown, J. M. et al. Conjugated linoleic acid induces human adipocyte delipidation: autocrine/paracrine regulation of MEK/ERK signaling by adipocytokines. J. Biol. Chem. 279, 26735-26747 (2004).
32. House, R. L. et al. Functional genomic characterization of delipidation elicited by trans-10, cis-12-conjugated linoleic acid (t10c12-CLA) in a polygenic obese line of mice, Physiol. Genomics 21, 351-361 (2005),
33. Van, R. L., Bayliss, C. E. & Roncari, D. A. Cytological and enzymological characterization of adult human adipocyte precursors in culture. J. Clin. Invest. 58, 699-704 (1976),
34. Negrel, R., Grimaldi, P. & Ailhaud, G. Establishment of preadipocyte clonal line from epididymal fat pad of ob/ob mouse that responds to insulin and to lipolytic hormones. Proc. Natl Acad. Sci. USA 75, 6054-6058 (1978).
35. Verges, B., Walter, T, & Cariou, B. Endocrine side effects of anti-cancer drugs: effects of anti-cancer targeted therapies on lipid and glucose metabolism. Eur. J. Endocrinol 170, R43-R55 (2014),
36. Zhang, Y. & Chua, S. Jr. Leptin function and regulation. Compr. Physiol 8, 351-369 (2017),
37. Leong, K. W., & Qiang, L. Targeting obesity: eliminating visceral fat. Columbia Engineering Magazine (spring 2022).
38. Higuchi, S. et al. Bile acid composition regulates GPR119-dependent intestinal lipid sensing and food intake regulation in mice. Gut 69, 1620-1628 (2020).
39. Brown, B. N. et al. Comparison of three methods for the derivation of a biologic scaffold composed of adipose tissue extracellular matrix, Tissue Eng. Part C Methods 17, 411-421 (2011).
40. Lee, M. J. & Fried, S. K. Optimal protocol for the differentiation and metabolic analysis of human adipose stromal cells. Methods Enzymol. 538, 49-65 (2014).
41. Lee, M. J., Gong, D. W., Burkey, B. F. & Fried, S. K. Pathways regulated by gliucocorticoids in omental and subcutaneous human adipose tissues: a microarray study. Am. J. Physiol. Endocrinol. Metab. 300, E571-E580 (2011).
42. Wan, Q., Huang, B., Li, T., Xiao, Y., He, Y., Du, W., Wang, B. Z., Dakin, G. F., Rosenbaum, M., Goncalves, M. D., Chen, S., Leong, K. W., & Qiang, L. Selective targeting of visceral adiposity by polycation nanomedicine. Nature Nanotechnology 17, 1311-21 (2022). Available online: www.nature.com/articles/s41565-022-01249-3.

It is to be understood that the methods of treating or preventing obesity, targeting adipose tissue, or delivery of treating agents, are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A method of treating or preventing obesity, comprising the step of administering to a patient in need thereof a pharmaceutically acceptable amount of polycationic polymer and derivatives.

2. The method of treating or preventing obesity as recited in claim 1, wherein the step of administering comprises intraperitoneal (IP) injection.

3. The method of treating or preventing obesity as recited in claim 1, wherein the polycationic polymer is P-G3, and the P-G3 is administered as lipophilic P-G3 nanoparticles.

4. The method of treating or preventing obesity as recited in claim 1, wherein the polycationic polymer is a PCL-g-PAMAM Denpol.

5. The method of treating or preventing obesity as recited in claim 3, wherein the method includes the step of first determining that the patient is likely to become obese, and the administering of P-G3 is intended to slow or prevent the development of obesity in the patient.

6. The method of treating or preventing obesity as recited in claim 3, wherein the lipophilic P-G3 nanoparticles are P-G3-Chol(5).

7. The method of claim 1, wherein the obesity is diet-induced obesity.

8. A method of targeting adipose tissue comprising administration of a suitable polycationic polymer or derivative as treatment or drug carrier.

9. The method of claim 8, wherein the method comprises treating a patient for obesity, diabetes, liver metabolic diseases, or aging.

10. The method of claim 8, wherein a treating agent is delivered in combination with the polycationic polymer or derivative.

11. The method of claim 10, where the treating agent comprises one or more fat-manipulating reagents, such as the following: aspirin, ibuprofen, celecoxib, indomethacin, ordiclofenac, pirfenidone, Nintedanib, dasatinib, navitoclax, quercetin, and fisetin.

12. The method of claim 8, wherein the method inhibits visceral adiposity, increases energy expenditure, prevents obesity, and/or alleviates associated metabolic dysfunctions.

13. The method of claim 8, wherein the method enhances visceral fat distribution.

14. The method of claim 8, wherein the method impedes adipocyte hypertrophic growth and reduces the size of adipocytes.

15. The method of claim 8, wherein the method promotes adipogenesis.

16. The method of claim 1, wherein the method inhibits visceral adiposity, increases energy expenditure, prevents obesity, and/or alleviates associated metabolic dysfunctions.

17. The method of claim 1, wherein the method enhances visceral fat distribution.

18. The method of claim 1, wherein the method impedes adipocyte hypertrophic growth and reduces the size of adipocytes.

19. The method of claim 1, wherein the method promotes adipogenesis.
